(19) United States
(12) Patent Application Publication
(10) Pub. No.: US 2025/0131332 A1
(43) Pub. Date: Apr. 24, 2025

(54) Systems and Methods for Bayesian Prognostic Covariate Adjustment

(71) Applicant: Unlearn.AI, Inc., San Francisco, CA (US)

(72) Inventors: Alyssa M. Vanderbeek, San Francisco, CA (US); Arman Sabbaghi, Martinez, CA (US); Jonathan Ryan Walsh, El Cerrito, CA (US); Charles Kenneth Fisher, Truckee, CA (US)

(73) Assignee: Unlearn.AI, Inc., San Francisco, CA (US)

(21) Appl. No.: 18/920,772

(22) Filed: Oct. 18, 2024

Related U.S. Application Data
(60) Provisional application No. 63/591,421, filed on Oct. 18, 2023.
(60) Provisional application No. 63/593,507, filed on Oct. 26, 2023.
(60) Provisional application No. 63/552,962, filed on Feb. 13, 2024.

(51) Int. Cl. G06N 20/00; G16H 10/20

(52) U.S. Cl. CPC G06N 20/00; G16H 10/20

(57) ABSTRACT

Systems and methods for Bayesian PROCOVA operations are illustrated. One embodiment includes a method for updating predictive models. The method trains a set of one or more generative models based on RCT data. The method defines a mixture prior distribution that includes: an informative component that follows an informative prior distribution defined, at least in part, on the RCT data; and a flat component that follows a flat prior distribution defined independently of the RCT data. The method generates, using the set of one or more generative models, predicted panel data for a plurality of digital subjects. The method derives a mixture posterior distribution corresponding to the unknown parameters of the set of one or more generative models, based on the predicted panel data. The method determines, based on at least one of the predicted panel data or the mixture posterior distribution, a set of one or more decision rules.

### CROSS-REFERENCE TO RELATED APPLICATIONS

The current application claims the benefit of and priority under 35 U.S.C. § 119(e) to U.S. Provisional Patent Application No. 63/591,421 entitled “Bayesian Prognostic Covariate Adjustment with Mixture,” filed Oct. 18, 2023, U.S. Provisional Patent Application No. 63/593,507 entitled “Bayesian Prognostic Covariate Adjustment with Mixture,” filed Oct. 26, 2023, and U.S. Provisional Patent Application No. 63/552,962 entitled “Bayesian PROCOVA v2.0 Method Proposal,” filed Feb. 13, 2024, the disclosures of which are hereby incorporated by reference in their entireties for all purposes.

### FIELD OF THE INVENTION

The present invention generally relates to prognostic models and, more specifically, the application of prognostic models to assessments of trial uncertainty.

### BACKGROUND

Randomized Controlled Trials (RCTs) are commonly used to assess the safety and efficacy of new treatments, including drugs and medical devices. In RCTs, subjects with particular characteristics are randomly assigned to one or more experimental groups receiving new treatments or to a control group receiving a comparative treatment (e.g., a placebo), and the outcomes from these groups are compared in order to assess the safety and efficacy of the new treatments.

Prognostic models are mathematical models that relate a subject's characteristics now to the risk of a particular future outcome, thereby allowing for RCTs to be efficiently represented. For example, Artificial Intelligence (AI) and Machine Learning (ML) algorithms may enable prognostic models to use historical data to create more efficient trials without introducing bias. When modelling RCTs in a medical context, prognostic models are used to compute prognostic scores, which correlate to the expected outcomes for participants with specific pre-treatment covariates if they receive specific control treatments.

### SUMMARY OF THE INVENTION

Systems and methods for Bayesian Prognostic Covariate Adjustment operations are illustrated. One embodiment includes a method for updating predictive models. The method receives, from at least one randomized controlled trial (RCT), RCT data, wherein the RCT data includes information on trial subjects from control arms of the at least one RCT. The method trains a set of one or more generative models based on the RCT data. The method defines a mixture prior distribution corresponding to unknown parameters of the set of one or more generative models. The mixture prior distribution includes: an informative component, wherein the informative component follows an informative prior distribution defined, at least in part, on the RCT data; and a flat component, wherein the flat component follows a flat prior distribution defined independently of the RCT data. The method generates, using the set of one or more generative models, predicted panel data for a plurality of digital subjects, wherein the predicted panel data for a given digital subject includes a plurality of predicted outcomes on at least one characteristic of the given digital subject in response to applying a treatment in a target RCT. The method derives a mixture posterior distribution corresponding to the unknown parameters of the set of one or more generative models, based on the predicted panel data. The method determines, based on at least one of the predicted panel data or the mixture posterior distribution, a set of one or more decision rules, wherein the set of one or more decision rules govern type-I estimates for the set of one or more generative models.

In a further embodiment, defining the mixture prior distribution includes computing a weighted combination of the informative component and the flat component. The informative component has a first weight and the flat component has a second weight. Moreover, deriving the mixture posterior distribution includes computing an updated first weight and an updated second weight.

In a still further embodiment, the weighted combination is a weighted sum. The first weight is selected from a weight parameter prior distribution that follows a Beta distribution. The first weight and second weight sum to 1.

In another embodiment, generating the predicted panel data, for each digital subject of the plurality of digital subjects, includes: determining, for the digital subject, at least one of: a baseline vector, wherein the baseline vector includes time-dependent measurements, corresponding to a specific time before a given RCT, that are based on the RCT data; or a covariate vector, wherein the covariate vector includes time-independent characteristics of the digital subject that are based on the RCT data. The generation further includes inputting the at least one of the baseline vector or the covariate vector into the set of one or more generative models.

In a further embodiment, generating the predicted panel data, for each digital subject of the plurality of digital subjects, further includes: obtaining, from the set of one or more generative models, a control outcome distribution, wherein the control outcome distribution describes potential values for an expected outcome if the digital subject were placed in a control group for the given RCT. The generation further includes determining the control outcome distribution, a score for the digital subject, wherein the score corresponds to the expected outcome. The generation further includes inputting, into the set of one or more generative models: a randomized treatment indicator, wherein the randomized treatment indicator corresponds to whether the digital subject is part of a treatment arm in the target RCT; and the score for the digital subject.

In still another embodiment, defining the informative prior distribution is based on: a first informative parameter selected from an Inverse Chi-Squared probability distribution, wherein the Inverse Chi-Squared probability distribution is defined based on the RCT data; and a second informative parameter selected from a Multivariate Normal probability distribution, wherein the Multivariate Normal probability distribution is defined based on the RCT data and the first informative parameter. Further, defining the flat prior distribution is based on: a first flat parameter selected from an Inverse Chi-Squared probability distribution, wherein the Inverse Chi-Squared probability distribution is defined based on a set of input flat values; and a second flat parameter selected from a Multivariate Normal probability distribution, wherein the Multivariate Normal probability distribution is defined based on the set of input flat values and the first flat parameter.

In a further embodiment, the second informative parameter is further defined based on at least one of: a discount factor, wherein the discount factor represents a discount to an amount of the RCT data used when training the set of one or more generative models; or a maximum estimate for an offset value, wherein the offset value represents a difference between a first bias measured for the RCT data and a second bias measured for the predicted panel data.

In a still further embodiment, the discount factor is determined based on a population size for the plurality of digital subjects; and the maximum estimate is linearly proportional to a square root of a variance in the offset value, wherein the variance in the offset value is determined based on bootstrap sampling of the RCT data to determine various values for the first bias.

In another embodiment, determining the set of one or more decision rules includes: estimating values for the unknown parameters, wherein: Gibbs sampling is used for estimating the values; and at least one of the unknown parameters relates to an effect of the treatment. The determination further includes deriving an uncertainty estimate corresponding to the estimated effect of the treatment. Determining the set of one or more decision rules is done based on the uncertainty estimate.

In another embodiment, the set of one or more generative models includes at least one of a Conditional Restricted Boltzmann Machine, a statistical model, a generative adversarial network, a recurrent neural network, a Gaussian process, an autoencoder, an autoregressive model, or a variational autoencoder.

One embodiment includes a non-transitory computer-readable medium including instructions that, when executed, are configured to cause a processor to perform a process for updating predictive models. The processor receives, from at least one randomized controlled trial (RCT), RCT data, wherein the RCT data includes information on trial subjects from control arms of the at least one RCT. The processor trains a set of one or more generative models based on the RCT data. The processor defines a mixture prior distribution corresponding to unknown parameters of the set of one or more generative models. The mixture prior distribution includes: an informative component, wherein the informative component follows an informative prior distribution defined, at least in part, on the RCT data; and a flat component, wherein the flat component follows a flat prior distribution defined independently of the RCT data. The processor generates, using the set of one or more generative models, predicted panel data for a plurality of digital subjects, wherein the predicted panel data for a given digital subject includes a plurality of predicted outcomes on at least one characteristic of the given digital subject in response to applying a treatment in a target RCT. The processor derives a mixture posterior distribution corresponding to the unknown parameters of the set of one or more generative models, based on the predicted panel data. The processor determines, based on at least one of the predicted panel data or the mixture posterior distribution, a set of one or more decision rules, wherein the set of one or more decision rules govern type-I estimates for the set of one or more generative models.

In a further embodiment, defining the mixture prior distribution includes computing a weighted combination of the informative component and the flat component. The informative component has a first weight and the flat component has a second weight. Moreover, deriving the mixture posterior distribution includes computing an updated first weight and an updated second weight.

In a still further embodiment, the weighted combination is a weighted sum. The first weight is selected from a weight parameter prior distribution that follows a Beta distribution. The first weight and second weight sum to 1.

In another embodiment, generating the predicted panel data, for each digital subject of the plurality of digital subjects, includes: determining, for the digital subject, at least one of: a baseline vector, wherein the baseline vector includes time-dependent measurements, corresponding to a specific time before a given RCT, that are based on the RCT data; or a covariate vector, wherein the covariate vector includes time-independent characteristics of the digital subject that are based on the RCT data. The generation further includes inputting the at least one of the baseline vector or the covariate vector into the set of one or more generative models.

In a further embodiment, generating the predicted panel data, for each digital subject of the plurality of digital subjects, further includes: obtaining, from the set of one or more generative models, a control outcome distribution, wherein the control outcome distribution describes potential values for an expected outcome if the digital subject were placed in a control group for the given RCT. The generation further includes determining the control outcome distribution, a score for the digital subject, wherein the score corresponds to the expected outcome. The generation further includes inputting, into the set of one or more generative models: a randomized treatment indicator, wherein the randomized treatment indicator corresponds to whether the digital subject is part of a treatment arm in the target RCT; and the score for the digital subject.

In still another embodiment, defining the informative prior distribution is based on: a first informative parameter selected from an Inverse Chi-Squared probability distribution, wherein the Inverse Chi-Squared probability distribution is defined based on the RCT data; and a second informative parameter selected from a Multivariate Normal probability distribution, wherein the Multivariate Normal probability distribution is defined based on the RCT data and the first informative parameter. Further, defining the flat prior distribution is based on: a first flat parameter selected from an Inverse Chi-Squared probability distribution, wherein the Inverse Chi-Squared probability distribution is defined based on a set of input flat values; and a second flat parameter selected from a Multivariate Normal probability distribution, wherein the Multivariate Normal probability distribution is defined based on the set of input flat values and the first flat parameter.

In a further embodiment, the second informative parameter is further defined based on at least one of: a discount factor, wherein the discount factor represents a discount to an amount of the RCT data used when training the set of one or more generative models; or a maximum estimate for an offset value, wherein the offset value represents a difference between a first bias measured for the RCT data and a second bias measured for the predicted panel data.

In a still further embodiment, the discount factor is determined based on a population size for the plurality of digital subjects; and the maximum estimate is linearly proportional to a square root of a variance in the offset value, wherein the variance in the offset value is determined based on bootstrap sampling of the RCT data to determine various values for the first bias.

In another embodiment, determining the set of one or more decision rules includes: estimating values for the unknown parameters, wherein: Gibbs sampling is used for estimating the values; and at least one of the unknown parameters relates to an effect of the treatment. The determination further includes deriving an uncertainty estimate corresponding to the estimated effect of the treatment. Determining the set of one or more decision rules is done based on the uncertainty estimate.

In another embodiment, the set of one or more generative models includes at least one of a Conditional Restricted Boltzmann Machine, a statistical model, a generative adversarial network, a recurrent neural network, a Gaussian process, an autoencoder, an autoregressive model, or a variational autoencoder.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a system for using linear models and digital twins to estimate treatment effects in accordance with several embodiments of the invention.

FIG. 2 illustrates uses for generative models in analyses of clinical trials performed in accordance with various embodiments of the invention.

FIG. 3 illustrates a process for designing a randomized controlled trial with prognostic effect estimation implemented in accordance with miscellaneous embodiments of the invention.

FIG. 4A illustrates a system for using generative models to estimate treatment effects in accordance with some embodiments of the invention.

FIG. 4B illustrates a system for borrowing information from digital twins to estimate treatment effects in accordance with many embodiments of the invention.

FIG. 5 illustrates a process for performing Bayesian prognostic covariate adjustments implemented in accordance with numerous embodiments of the invention.

FIG. 6 illustrates a process for determining an additive mixture prior in accordance with some embodiments of the invention.

FIG. 7 illustrates a process for determining a mixture prior in accordance with multiple embodiments of the invention.

FIG. 8 illustrates a treatment analysis system that determines treatment effects in accordance with some embodiments of the invention.

FIG. 9 illustrates a treatment analysis element that executes instructions to perform processes that determine treatment effects in accordance with various embodiments of the invention.

FIG. 10 illustrates a treatment analysis application for determining treatment effects in accordance with numerous embodiments of the invention.

### DETAILED DESCRIPTION

Systems and methods implemented in accordance with various embodiments of the invention may be directed to Bayesian Prognostic Covariate Adjustment (Bayesian PROCOVA) operations. In this disclosure, Bayesian PROCOVA may refer to a process that implements Bayes' rule to refine prognostic model distributions used to estimate treatment effects. Systems may thereby implement Bayesian PROCOVA by performing covariate adjustment via prognostic model outcomes, leveraging previously-obtained information in the analysis of RCTs. Naturally, the incorporation of Bayes' rule will depend on the application of prior distributions (representing estimates of uncertain parameters used before any new observations are obtained) and posterior distributions (representing the influence of obtained observations on the estimates used for the uncertain parameters).

Systems and methods in accordance with various embodiments of the invention may utilize historical data from (control arms) of previous RCTs to produce an (additive mixture) prior that is completely pre-specifiable, and therefore does not involve any information from soon-to-be implemented RCTs. Additive mixture priors may include but are not limited to an informative prior probability distribution based on the aforementioned control data, a non-informative prior distribution, and at least one weight parameter also following a prior distribution. Additionally or alternatively, systems may depend on user-specified parameters representing attributes including but not limited to anticipated changes in bias (digital twin offsets); and/or constants that limit the impact of historical data (discount factors). Various implementations of Bayesian PROCOVA, as applied in accordance with some embodiments of the invention are discussed below.

### I. Generative Models

Turning now to the drawings, systems and methods implemented in accordance with many embodiments of the invention may be implemented to yield efficient estimators and tests for treatment effects observed in randomized controlled trials (RCTs). In obtaining treatment effect inferences from such methods, systems may be configured to incorporate predictions from generative artificial intelligence (AI) algorithms into aspects of RCT regression analyses, including but not limited to adjustment of covariates (i.e., independent variables) for the regression analyses. In accordance with many embodiments of the invention, adjustments (e.g., in the form of determined coefficients to the known and/or derived values) may be facilitated through methodology including but not limited to Prognostic Covariate Adjustment (PROCOVA) methods. PROCOVA methodology is disclosed in Unlearn.AI, Inc., “PROCOVA™ Handbook for the Target Trial Statistician.” Ver. 1.0, European Medicines Agency, incorporated herein by reference. By using generative AI, systems can effectively apply determined relationships (between the variation in the outcomes and at least some of the covariates) to produce outputs including but not limited to scalar features that can be optimized to explain variation in observed outcomes.

In accordance with some embodiments of the invention, training generative AI algorithms on historical control data (e.g., data obtained from previous RCTs) may enable the construction and updating of digital twin generators (DTGs). DTGs implemented in accordance with numerous embodiments of the invention may correspond to entities including but not limited to RCT participants and/or utilize neural network architectures that can learn conditional generative models of patient trajectories (e.g., based on historical data). For example, DTGs may utilize the baseline covariates of participants (e.g., attributes observed prior to running RCTs) to generate probability distributions for potential control outcomes of the participants (digital twins). Summaries of the probability distribution from the DTGs may effectively predict trial outcomes. Additionally or alternatively, adjusting for covariates via regression can thus improve the quality of output including but not limited to treatment effect inferences, RCT designs, and/or decision rules for treatments.

Data sampled from generative models in accordance with a number embodiments of the invention may be referred to as ‘digital subjects’ throughout this description. In many embodiments, digital subjects can be generated to match given statistics of the treatment groups at the beginning of the study. Digital subjects in accordance with numerous embodiments of the invention can be generated for each subject in a study and the generated digital subjects can be used as digital twins for a counterfactual analysis. In various embodiments, generative models can be used to compute measures of response (that can be individualized to each patient and/or used to assess the effects of the treatments. In many embodiments of the invention, estimates for treatment effects may be derived using linear regression models. Moreover, as suggested above, systems and methods in accordance with several embodiments of the invention can (additionally or alternatively) correct for bias that may be introduced by incorporating generated digital subject data.

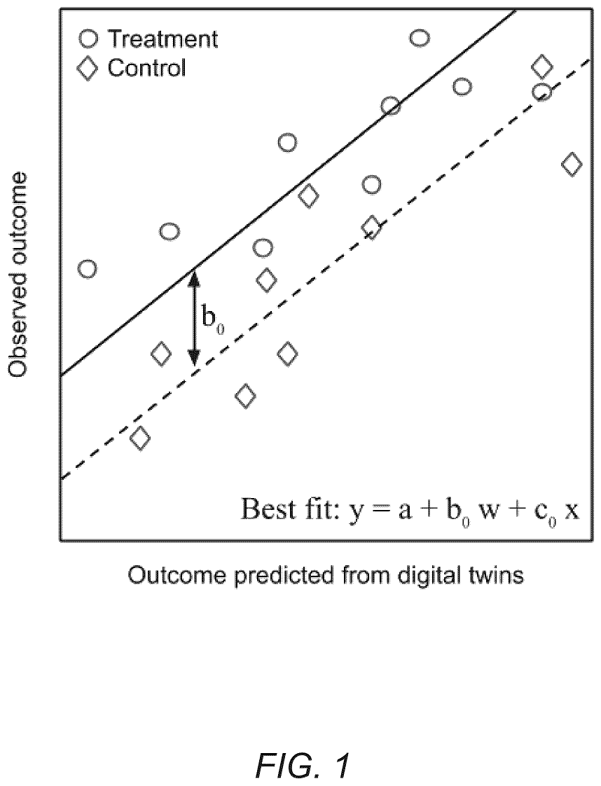

An example of the uses of linear models and digital twins in estimating treatment effects in accordance with an embodiment of the invention is illustrated in FIG. 1. This drawing depicts the concept using a simple analysis of a continuous outcome. The x-axis represents the prediction for the outcome from the digital twins, and the y-axis represents the observed outcome of the subjects in the RCT. A linear model is fit to the data from the RCT, adjusting for the outcome predicted from the digital twins. When no interactions are included, then two parallel lines are fit to the data: one to the control group and one to the treatment group. The distance between these lines is an estimate for the treatment effect. As will be shown below, in accordance with many embodiments of the invention, Bayesian methods may be used to analyze the linear models.

Systems configured in accordance with multiple embodiments of the invention may endeavor to yield optimal treatment effect inferences in cases of homoskedasticity: i.e., consistency of outcome variance conditional on the covariates. Specifically, the case of nonconstant variance of outcomes (i.e., heteroskedasticity), adjustment methods may not satisfactorily improve treatment effect inferences. Systems and methods in accordance with several embodiments of the invention may enable (treatment effect-optimizing) adjustments with regard to various statistical terms including but not limited to standard errors. Covariate adjustments implemented in accordance with miscellaneous embodiments of the invention may involve regression models that include the covariates associated with the outcomes and/or the treatment (i.e., treatment indicator variable) as predictor variables.

In accordance with some embodiments, adjustments may be based on the incorporation of predictor variables including but not limited to prognostic scores determined by models of expected outcomes, and variances of the outcomes. In accordance with many embodiments of the invention, prognostic scores may refer to mean/average values of prognostic models including but not limited to digital twins. Additionally or alternatively, variances of outcomes may refer to variances of prognostic models including but not limited to digital twins, a specific classification of AI-based prognostic models. In this disclosure, digital twins may refer to digital representations of physical objects, processes, services, and/or environments with the capacity to behave like their counterparts in the real world. In the context of drug and medical studies, digital twins can take the form of representations of the range of potential control (placebo) outcomes of particular clinical trial participants given their baseline covariates/characteristics.

As mentioned above, systems and methods configured in accordance with various embodiments of the invention may be directed to determining the treatment effects of RCTs. RCT data can include panel data collected from subjects of RCTs and/or can be supplemented with generated subject data. Generated subject data in accordance with a number of embodiments of the invention can include (but are not limited to) digital subject data and/or digital twin data obtained from generative models.

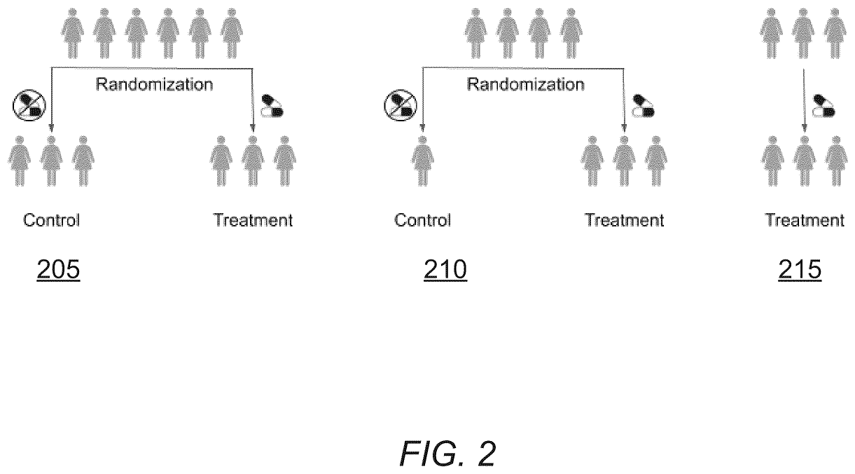

Examples of uses for generative models in the analysis of clinical trials in accordance with various embodiments of the invention are illustrated in FIG. 2. The first example 205 illustrates that generative models, digital subjects, and/or digital twins can be used to increase the statistical power of traditional randomized controlled trials. In the second example 210, generated data is used to decrease the number of subjects required to be enrolled in the control group of a randomized controlled trial. The third example 215 shows that generated data can be used in the external comparator arm of a single-arm trial.

In an RCT, a group of subjects with particular characteristics are randomly assigned to one or more experimental groups receiving new treatments and/or to a control group receiving a comparative treatment (e.g., a placebo), and the outcomes from these groups can be compared in order to assess the safety and efficacy of the new treatments. Without loss of generality, an RCT can be assumed to include i=1, . . . , N human subjects. These subjects are often randomly assigned to a control group and/or to a treatment group such that the probability of being assigned to the treatment group is the same for each subject regardless of any unobserved characteristics. The assignment of subject i to a group is represented by a treatment indicator variable wᵢ. For example, in a study with two groups wᵢ=0 if subject i is assigned to the control group and wᵢ=1 if subject i is assigned to the treatment group. The number of subjects assigned to the treatment group is N_{T}=Σᵢwᵢ and the number of subjects assigned to the control group is N_{C}=N−N_{T}.

In various embodiments, each subject i=1, . . . , N in an RCT can be described by a vector xᵢ(t) of covariate variables xᵢⱼ(t) at time t. In this description, the notation xᵢ={xᵢ(t)}[ₜ₌₁^{T} denotes the panel of data from subject i and the notation xᵢ(0) and/or x₀ to denote the vector of data taken at time zero. An RCT is often concerned with estimating how a treatment affects an outcome yᵢ=ƒ(xᵢ). For each pair of participant i and treatment wᵢ, systems implemented in accordance with some embodiments can define the potential outcome yᵢ as the endpoint value that would be observed at a specified time-point after treatment assignment. The function ƒ(·) describes the combination of variables being used to assess the outcome of the treatment. Variables in accordance with a number of embodiments of the invention can include (but are not limited to) simple endpoints based on the value of a single variable at the end of the study, composite scores constructed from the characteristics of a patient at the end of the study, and/or time-dependent outcomes such as rates of range and/or survival times, among others. Approaches in accordance with various embodiments of the invention can be applied to analyze the effect of treatments on one or more outcomes, such as (but not limited to) those related to the efficacy and safety of the treatment.

Each subject has two potential outcomes. When the subject is assigned to the control group wᵢ=0, then yᵢ(0) would be the observed potential outcome. By contrast, when the subject is assigned to receive treatment wᵢ=1, then yᵢ(1) would be the observed potential outcome. In practice, subjects may be assigned to one of the treatment arms such that the observed outcome is Yᵢ=yᵢ(0)(1−wᵢ)+wᵢyᵢ(1). In accordance with various embodiments of the invention, Bayesian PROCOVA may be formulated under the Neyman-Rubin Causal Model, and therefore, follow assumptions including but not limited to the Stable Unit-Treatment Value Assumption (indicating there exists no hidden varieties of treatment for any participant, and the potential outcome for a participant is not a function of treatments assigned to other participants but is solely a function of their treatment assignment). Under this assumption, the potential outcomes (i.e., yᵢ(0), yᵢ(1)) of each participant may be well-defined. Potential outcomes in accordance with many embodiments of the invention can include various measurements, such as, but not limited to conditional average treatment effect:

$$τ ( {x}_{0} ) = E [ w = 1 , {x}_{0} ] - E [ w = 0 , {x}_{0} ] \qquad ( i )$$

and/or the average treatment effect:

$$τ = E [ τ ( {x}_{0} ) ] = E [ y \vert w = 1 ] - E [ y \vert w = 0 ] . \qquad ( ii )$$

Processes in accordance with several embodiments of the invention can estimate these quantities with high accuracy and precision and/or can determine decision rules for declaring treatments to be effective that have low error rates.

It can be expensive, time-consuming and, in some cases, unethical to recruit human subjects to participate in RCTs. As a result, a number of methods have been developed for using external control arms to reduce the number of subjects required for an RCT. These methods typically fall into two buckets referred to as ‘historical borrowing’ and ‘external control’. Historical borrowing refers to incorporating data from the control arms of previously completed trials into the analysis of a new trial. Typically, historical borrowing applies Bayesian methods using prior distributions derived from the historical dataset. Such methods can be used to increase the power of a randomized controlled trial, to decrease the size of the control arm, and/or even to replace the control arm with the historical data itself (i.e., an ‘external control arm’). Some examples of external control arms include control arms from previously completed clinical trials (also called historical control arms), patient registries, and data collected from patients undergoing routine care (called real-world data).

Digital subject data utilized in accordance with many embodiments of the invention may be generated using generative models. Generative models in accordance with certain embodiments of the invention can be trained to generate potential outcome data based on characteristics of individuals and/or populations. Digital subject data in accordance with several embodiments of the invention can include (but are not limited to) panel data, outcome data, etc. In numerous embodiments, generative models can be trained directly on a specific outcome p(y|x₀). For example, if a goal of using the generative model is to increase the statistical power for the primary analysis of a randomized controlled trial then it may be sufficient (but not necessary) to only use a model of p(y|x₀).

Alternatively, or conjunctively, generative models may be trained to generate panel data that can be used in the analysis of a clinical trial. Data for a subject in a clinical trial typically includes a panel; that is, it describes the observed values of multiple characteristics at multiple discrete time points (e.g., visits to the clinical trial site). For example, if a goal of using the generative model is to reduce the number of subjects in the control group of the trial, or as an external comparator for a single-arm trial, then generated panel data in accordance with many embodiments of the invention can be used to perform many or all of the analyses of the trial.

In several embodiments, generative models can include (but are not limited to) traditional statistical models, generative adversarial networks, recurrent neural networks, Gaussian processes, autoencoders, autoregressive models, variational autoencoders, and/or other types of probabilistic generative models. For example, processes in accordance with several embodiments of the invention can use sequential models such as (but not limited to) Restricted Boltzmann Machines for the full joint distribution of the panel data, p(X), from which any outcome can be computed.

Systems configured in accordance with some embodiments apply machine learning methods to create simulated subject records. In addition to data from RCTs, generative models in accordance with several embodiments of the invention can link the baseline characteristics and the control potential outcomes through joint probability distributions and conditional probability distributions. Note that a model of the joint distribution will also provide a model of the conditional distribution, but the converse is not true.

In several embodiments, simulated subject records can be sampled from probabilistic generative models that can be trained on various data, such as (but not limited to) one or more of historical, registry, and/or real-world data. Such models can allow one to extrapolate to new patient populations and study designs.

In some embodiments, generative models may create data in a specialized format—either directly or indirectly—such as the Study Data Tabulation Model (SD™) and/or the Neyman-Rubin Causal Model to facilitate seamless integration into standard workβ0ws. In a variety of embodiments, generating entire panels of data can be attractive because many of the trial outcomes (such as primary, secondary, and exploratory endpoints as well as safety information) can be analyzed in a parsimonious way using a single generative model.

Systems and methods in accordance with numerous embodiments of the invention can provide various approaches for incorporating data from probabilistic generative models into the analysis of RCTs. In numerous embodiments, such methods can be viewed as borrowing from a model, as opposed to directly borrowing from a historical dataset. As generative models, from which data can be borrowed, may be biased (for example, due to incorrect modelling assumptions), systems and methods in accordance with a number of embodiments of the invention can account for these potential biases in the analysis of RCTs. Generative models in accordance with various embodiments of the invention can provide control over the characteristics of each simulated subject at the beginning of the study. For example, processes in accordance with various embodiments of the invention can create one or more digital twins for each human subject in the study. Processes in accordance with certain embodiments of the invention can incorporate digital twins to increase statistical power and can provide more individualized information than traditional study designs, such as study designs that borrow population-level information and/or that use nearest neighbor matches to patients in historical or real-world databases.

Some systems implemented in accordance with multiple embodiments of the invention can estimate treatment effects using models while adjusting for covariates. For example, systems may perform a regression of the final outcome in the trial against the treatment indicator and a measure of disease severity at the start of the trial. As long as the covariate was measured before the treatment was assigned in a randomized controlled trial, then adjusting for the covariate will not bias the estimate for the treatment effect in a frequentist analysis. When using covariate adjustments, the statistical power is a function of the correlation between the outcome and the covariate being adjusted for; the larger the correlation, the higher the power.

In many embodiments, processes can estimate treatment effects by training new generative models including but not limited to treatment models using the data from the treatment group, p_{θ}_{_{J}}(w₁), and a control model using the data from the control group, p_{θ}_{_{J}}(w₀). In a variety of embodiments, full panels of data from an RCT can be used to train generative models to create panels of generated data. Such processes can allow for the analysis of many outcomes (including but not limited to primary, secondary, and exploratory efficacy endpoints as well as safety information) by comparing the trained treatment models against trained control models. For simplicity, the notation p(y,x₀) will be used instead of p(X), with the understanding that the former can always be obtained from the latter by generating a panel of data X and then computing a specific outcome y=ƒ(X) from the panel.

As mentioned above, generative models for the control condition (e.g., a Restricted Boltzmann Machine) can be trained on historical data from previously completed clinical trials. Then, two new generative models for the control and treatment groups can be obtained by solving minimization problems:

$${min}_{{θ}_{{J}_{0}}} \{ - {\sum }_{i} ( 1 - {w}_{i} ) log {p}_{{θ}_{{J}_{0}}} ( {Y}_{i} , {x}_{0 , i} \vert {w}_{0} ) + {λ}_{0} D ( {p}_{{θ}_{{J}_{0}}} , {p}_{{θ}_{J}} ) \} \qquad ( iii )$$

$${min}_{{θ}_{{J}_{1}}} \{ - {\sum }_{i} {w}_{i} log {p}_{{θ}_{{J}_{1}}} ( {Y}_{i} , {x}_{0 , i} \vert {w}_{1} ) + {λ}_{1} D ( {p}_{{θ}_{{J}_{1}}} , {p}_{{θ}_{J}} ) \} \qquad ( iv )$$

in which λ₀ and λ₁ are prior parameters that describe how well pre-trained generative models describe the outcomes in the two arms of the RCT, and D( [symbol] ) is a measure of the difference between the generative models such as (but not limited to) the Kullback-Leibler divergence.

The estimate for the treatment effect can then be computed as:

$$\hat{τ} = \int dy {dxyp}_{{θ}_{{J}_{1}}} ( y , x \vert {w}_{1} ) - \int dy {dxyp}_{{θ}_{{J}_{0}}} ( y , x \vert {w}_{0} ) . \qquad ( v )$$

In several embodiments, treatment effects can be computed by drawing samples from the control and treatment models and comparing the distributions of the samples. Processes in accordance with some embodiments of the invention can further tune the computation of treatment effects by adjusting for the uncertainty in treatment effect estimates. In several embodiments, the uncertainty in treatment effect estimates (σ_{\hat{τ}}) can be obtained using a bootstrap by repeatedly resampling the data from the RCT (with replacement), training the updated generative models, and computing an estimate for the treatment effect; the uncertainty is the standard deviation of these estimates. In a number of embodiments, point estimates for the treatment effect and the estimate for its uncertainty can be used to perform a hypothesis test in order to create a decision rule.

In numerous embodiments, processes can begin with a distribution π(θ_{J}) for the parameters of the generative model (e.g., obtained from a Bayesian analysis of historical data). Then, posterior distributions for θJ₀ and θ_{J}_{₁} can be estimated by applying Bayes rule:

$$log π ( {θ}_{{J}_{0}} ) = constant + {\sum }_{i} ( 1 - {w}_{i} ) {p}_{{θ}_{{J}_{0}}} ( {Y}_{i} , {x}_{i} \vert {w}_{0} ) + {λ}_{0} log π ( {θ}_{J} ) ( iv log π ( {θ}_{{J}_{0}} ) = constant + {\sum }_{i} {w}_{i} {p}_{{θ}_{{J}_{1}}} ( {Y}_{i} , {x}_{i} \vert {w}_{0} ) + {λ}_{0} log π ( {θ}_{J} ) . \qquad ( vi )$$

In certain embodiments, point estimates for the treatment effect can be calculated as the mean of the posterior distribution

$$\hat{τ} = \int dy dxd {θ}_{{J}_{1}} {yp}_{{θ}_{{J}_{0}}} ( y , x \vert {w}_{1} ) π ( {θ}_{{J}_{1}} ) - \int dy dxd {θ}_{{J}_{0}} {yp}_{{θ}_{{J}_{0}}} ( y , x \vert {w}_{0} ) π ( {θ}_{{J}_{1}} ) , \qquad ( vii )$$

where the uncertainty is the variance of the posterior distribution:

$${δ}^{2} τ = \int dy dxd {θ}_{{J}_{1}} {y}^{2} {p}_{{θ}_{{J}_{1}}} ( y , x \vert {w}_{1} ) π ( {θ}_{{J}_{1}} ) - \int dy dxd {θ}_{{J}_{0}} {y}^{2} {p}_{{θ}_{{J}_{0}}} ( y , x \vert {w}_{0} ) π ( {θ}_{{J}_{1}} ) - {\hat{τ}}^{2} . \qquad ( viii )$$

As above, point estimates for the treatment effect and estimates for their uncertainty can be used to perform a hypothesis test in order to create a decision rule in accordance with certain embodiments of the invention. Processes in accordance with a variety of embodiments of the invention can train generative models, in order to estimate treatment effects that are conditioned on the baseline covariates x₀.

It can be difficult to determine the operating characteristics of a decision rule based on these methods. Specifically, extensive simulations can be required in order to estimate the type-I error rate (i.e., the probability that an ineffective treatment would be declared to be effective) and the type-II error rate (i.e., the probability that an effective treatment would be declared ineffective). Well-characterized operating characteristics are required for many applications of RCTs and, as a result, this approach is often impractical. Generative models that rely on modern machine-learning techniques are typically computationally expensive to train. As a result, using the bootstrap and/or Bayesian methods to obtain uncertainties required to formulate reasonable decision rules can be quite challenging.

As shown above, systems and methods in accordance with numerous embodiments of the invention may determine treatment effects for RCTs using generated digital subject data. Generative models in accordance with many embodiments of the invention can be incorporated into the analysis of an RCT in a variety of different ways for various applications. In many embodiments, generative models can be used to estimate treatment effects by training separate generative models based on data from the control and treatment arms of previous trials. Processes in accordance with many embodiments of the invention can use generative models to generate digital subjects to supplement control arms in RCTs. In certain embodiments, processes can use generative models to generate digital twins for individuals in the control and/or treatment arms. Generative models in accordance with numerous embodiments of the invention may be used to define individualized responses to treatment. Various methods for determining treatment effects in accordance with various embodiments of the invention are described in greater detail herein.

### II. Prognostic Covariate Adjustment (PROCOVA)

Covariate adjustments for RCTs implemented in accordance with multiple embodiments of the invention may effectively refer to transformations of covariates. In particular, transformations that can yield significant variance reduction for the treatment effect estimator may include but are not limited to expected control outcomes conditional on the covariates. The application of generative models (including but not limited to optimized AI algorithms trained on historical control data) to define transformations can enable systems configured in accordance with multiple embodiments of the invention to obtain valid, efficient, and powerful treatment effect inferences via singular adjustments.

As such, in accordance with many embodiments of the invention, PROCOVA methods may be configured to use historical data and prognostic modelling to decrease the uncertainty associated with treatment effect estimates. Systems and methods in accordance with certain embodiments of the invention, when following PROCOVA processes, may implement least squares regression analysis of RCTs. Additionally or alternatively, PROCOVA processes may use heteroskedasticity-consistent (HC) standard errors to quantify the uncertainty associated with treatment effect estimators.

### A. Generalized PROCOVA

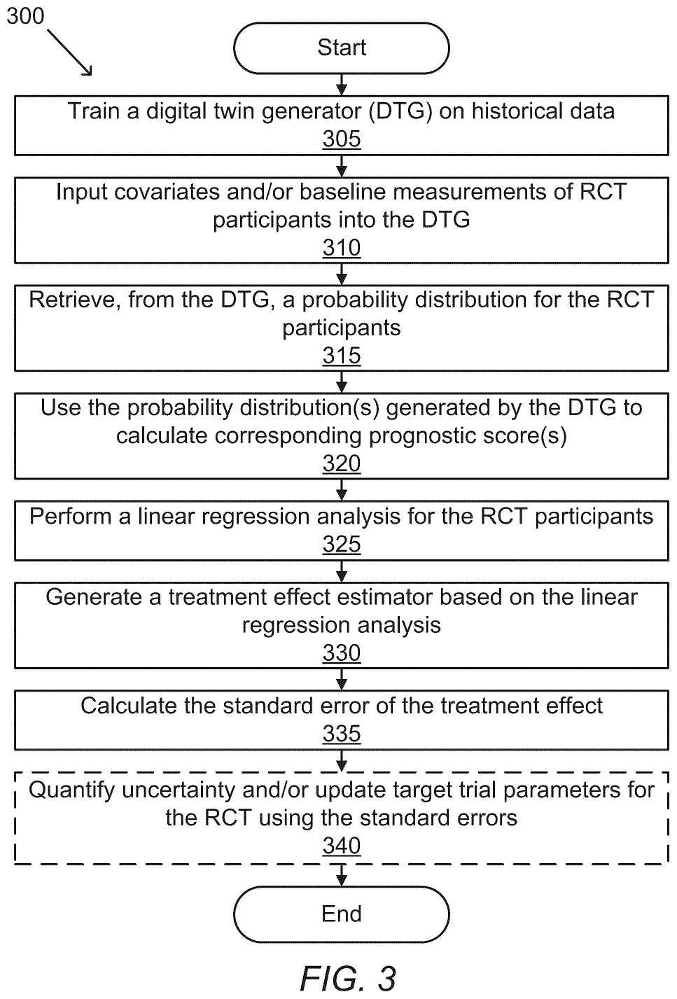

An example of the PROCOVA process applied, in accordance with multiple embodiments of the invention, to summarizing linear regression analysis of RCTs, is illustrated in FIG. 3. Process 300 trains (305) a digital twin generator (DTG) on historical control data. In accordance with many embodiments of the invention, historical control data may include but is not limited to control arm data from historical control arms of previous RCT trials. In some embodiments of the invention, training (305) DTGs may not involve any RCT (e.g., treatment) data. In doing so, the DTGs may be completely prespecified. Process 300 inputs (310) covariates and/or baseline measurements of RCT participants into the DTG. In accordance with some embodiments of the invention, the sole inputs for trained DTGs may be participant covariates and baseline measurements xᵢ. As such, they may avoid introducing any bias in treatment effect inferences for the RCT. In various embodiments, participant covariates may take the form of vectors of time-independent background variables including but not limited to race, sex, disability, and/or genetic profile. Additionally or alternatively, baseline measurements may incorporate various types of (e.g., time-dependent) information known before and/or at the start of prediction attempts. In particular, such characteristics may be used to answer: “Given a subject's baseline measurements, how will those characteristics evolve in time?” As such, in accordance with some embodiments, baseline covariates for individual clinical trials may include but are not limited to T-cell count, bone density, and/or BMI.

Process 300 retrieves (315), from the DTG, probability distribution(s) for the RCT participants. In accordance with many embodiments, the probability distributions of the DTGs may represent the potential outcomes for the RCT participants, if they were assigned to the control group of an RCT. Probability distributions generated in accordance with certain embodiments of the invention may consider each participant's fixed timepoint post-treatment assignment. Process 300 may represent the probability distributions by the cumulative distribution function (CDF) F_{i,0}: [symbol] →(0,1).

Process 300 uses (320) the probability distribution(s) (i.e., CDF, F_{i,0}) generated by the DTG to calculate corresponding prognostic score(s) (e.g., mᵢ=∫_{-∞}^{∞}rdF_{i,0}(r)) for the associated RCT participant. In accordance with various embodiments of the invention, prognostic scores may be calculated via Monte Carlo samples (i.e., by obtaining independent samples from each distribution F_{i,0} and calculating the averages of the respective samples). In doing so, process 300 may effectively summarize a large number of covariates into a scalar that is correlated with the observed outcomes. Additionally or alternatively, the prognostic scores may be calculated for all RCT participants independently of the arm of the RCT they end up assigned to (i.e., μᵢ being independent of wᵢ).

Process 300 performs (325) a linear regression analysis for the RCT participants based on the prognostic scores. In accordance with numerous embodiments, regression analysis may be represented in matrix form. For example, there may be N participants in a prospective RCT such that a first vector is an N×1 vector, representing the treatment effect (i.e., outcome to be extrapolated by the regression analysis) for each participant. In many embodiments of the invention, the linear regression analysis for each participant i may be based on vectors of predictors vᵢ. In addition to the prognostic scores (mᵢ), the vector of predictors for participants may include but is not limited to treatment indicators (wᵢ). Performing (325) linear analyses in accordance with various embodiments of the invention may include but is not limited to performing weighted linear analyses (i.e., Weighted PROCOVA) as described below. RCTs configured in accordance with many embodiments of the invention may apply treatments (i.e., wᵢ) randomly. Additionally or alternatively, the treatments may be applied in a manner independent to both pre-treatment covariates (i.e., xᵢ) and the potential outcomes. In accordance with certain embodiments, potential outcomes may include but are not limited to yᵢ⁽⁰⁾, which can represent the potential outcome(s) when participant i is assigned to a control group, and yᵢ⁽¹⁾, when participant i is assigned to a treatment group.

Process 300 generates (330) a treatment effect estimator (\hat{β}₁) based on the linear regression analysis. As indicated above, treatment effect estimators (or PROCOVA) in accordance with many embodiments of the invention presume a working model Yᵢ=β₀+β₁wᵢ+β₂μᵢ+ϵᵢ where Yᵢ, wᵢ, and μᵢ are a subject's outcome, treatment status, and prognostic score, respectively and ϵᵢ is a noise term. This model can be fit via ordinary least-squares and the value of β₁ can be taken as the point estimate of the treatment effect, \hat{β}₁. In several embodiments, the linear regression analyses may produce fitted regression models used for producing the treatment effect estimator(s). The impact of the treatment effect estimators is expounded upon in Schuler, A. et al. (2021) “Increasing the efficiency of randomized trial estimates via Linear Adjustment for a prognostic score,” The International Journal of Biostatistics, 18(2), pp. 329-356. Available at: https://doi.org/10.1515/ijb-2021-0072, incorporated by reference in its entirety.

Process 300 calculates (335) the standard error of the treatment effect. The standard error of treatment effects calculated in accordance with a number of embodiments of the invention may take on different forms including but not limited to heteroskedasticity-consistent (HC) standard errors (e.g., HC0, HC1, HC2, HC3). Additional prospective steps for computing HC standard errors are disclosed in Romano, J. P. and Wolf, M. (2017) “Resurrecting weighted least squares,” Journal of Econometrics, 197(1), pp. 1-19, Available at: https://doi.org/10.1016/j.jeconom.2016.10.003, incorporated by reference in its entirety.

In accordance with many embodiments of the invention, HC errors may be calculated to follow regulatory guidance standards regarding covariate adjustments for RCTs, including but not limited to those determined by the European Medicines Agency and the Food and Drug Administration (e.g., European Medicines Agency (2015). Guideline on Adjustment for Baseline Covariates in Clinical Trials; Food and Drug Administration, US Department of Health and Human Services, Center for Drug Evaluation and Research (CDER), and Center for Biologics Evaluation and Research (CBER) (2023). Adjusting for Covariates in Randomized Clinical Trials for Drugs and Biological Products: Guidance for Industry) incorporated by reference in their entireties.

In accordance with certain embodiments of the invention, the calculated (335) standard error may be used for various applications. Process 300 may quantify (340) uncertainty for with the RCT using the standard errors. Additionally or alternatively, process 300 may update target trial parameters for the RCT according to any quantified uncertainty. In accordance with many embodiments of the invention, prognostic model estimates for treatment effects and estimates for their uncertainty can be used to perform hypothesis tests in order to create decision rules that guide target trials.

While specific processes for determining treatment effects are described above in FIG. 3, any of a variety of processes can be utilized to implement randomized controlled trials as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

Defined in terms of the distributions of covariates and potential outcomes, covariate adjustment with prognostic scores can consistently yield smaller mean squared errors compared to the other methods, across multiple different settings. These observations demonstrate theoretical results that PROCOVA can minimize asymptotic variance among a class of estimators, the uncertainty in the treatment effect estimator may be minimized when the prognostic model predicts the participants' control potential outcomes, and/or gains can be realized in efficiency even with imperfect prognostic models/in the presence of heterogeneous effects. The observations also establish that PROCOVA may decrease the variance of the treatment effect estimators proportional to the squared correlation of the prognostic score with the outcome. In doing so, PROCOVA may guarantee unbiasedness, control of Type-I error rates, and/or desirable confidence interval coverage.

PROCOVA can unite AI and historical control data to decrease uncertainty in treatment effect inferences for RCTs. For example, DTGs implemented in accordance with certain embodiments of the invention may be implemented by a variety of mathematical and/or computational means. Additionally or alternatively, a priori variance reduction and/or power boosts may cast in terms of goodness-of-fit metrics for DTGs on validation data. The benefits of adjusting via the prognostic score(s) can increase as functions of the correlation(s) between the prognostic score(s) and the outcomes. Moreover, by adjusting based solely on the prognostic score(s) (instead of the entire predictor vector vᵢ), systems operating in accordance with numerous embodiments of the invention can use fewer degrees of freedom, which in turn can increase power (e.g., when M is large). PROCOVA implemented in accordance with many embodiments of the invention can be semiparametric efficient when the treatment effects are constant, the historical data follows the same distributions as the trial control arms, and/or the prognostic models improve with the amount of historical data. This implies that the power of a trial using PROCOVA will be greater than or equal to the power of any other trial design that controls the Type-I error rate.

As indicated above, treatment effect estimators (or PROCOVA) in accordance with many embodiments of the invention can presume a working model following (but not limited to) Yᵢ=β₀+β₁wᵢ+β₂μᵢ+ϵᵢ where Yᵢ, wᵢ, and μᵢ are a subject's outcome, treatment status, and prognostic score, respectively and ϵᵢ is a noise term (e.g., ϵᵢ˜N(0, σ²)). This model can be fit via ordinary least-squares and the value of β₁ can be taken as the point estimate of the treatment effect, \hat{β}₁. Under such formulations, β₀ can be interpreted as the bias of the average of the prognostic scores (\hat{β}₁) in predicting the endpoint of a participant in the control group. Additionally or alternatively, β₂ can represent a determination of how the bias varies with μᵢ. In several embodiments, treatment effects can be determined by fitting generalized linear models (GLMs) to the generated digital subject data and/or the RCT data. In a number of embodiments, multilevel GLMs can be set up so that the parameters (e.g., the treatment effect) can be estimated through Bayesian approaches, focusing on the posterior probability Prob(β₀≥0|data, prior), as described below. As in a typical Bayesian analysis, treatments can be declared effective when Prob(β₀≥0|data, prior) exceeds a pre-specified threshold in accordance with a number of embodiments of the invention.

A treatment effect estimate can be declared to be “statistically significant” at level α if a p<α where p=2*(min{Φ(\hat{β}₁/\hat{v}), (1−Φ(\hat{β}₁/\hat{v})} is the two-sided p-value, v is the standard error of β₁, and Φ denotes the CDF of the standard normal density. The assumption-free asymptotic sampling variance \hat{v}² ≡Var[\hat{β}₁] of the treatment effect

$${v}^{2} = \frac{{\hat{σ}}_{0}^{2}}{{n}_{0}} + \frac{{\hat{σ}}_{1}^{2}}{{n}_{1}} - \frac{{n}_{0} {n}_{1}}{{n}_{0} + {n}_{1}} {( \frac{{\hat{ρ}}_{0} {\hat{σ}}_{0}}{{n}_{1}} + \frac{{\hat{ρ}}_{1} {\hat{σ}}_{1}}{{n}_{0}} )}^{2} \qquad ( ix )$$

in which

$${\hat{ρ}}_{w} = \frac{Cov [ {Y}_{w} , μ ]}{\sqrt{Var [ μ ] Var [ {Y}_{w} ]}}$$

(where Y_{w} denotes potential outcomes under treatment w=1 and control w=0, \hat{σ}_{w}²=Var[Y_{w}], n₀ and n₁ are the number of enrolled control and treated subjects).

The probability that p<α when, in reality, the treatment effect is β₁ is given by

$$Power = Φ ( {Φ}^{- 1} ( α / 2 ) + \frac{{β}_{1}}{v} ) + Φ ( {Φ}^{- 1} ( α / 2 ) - \frac{{β}_{1}}{v} ) . \qquad ( x )$$

To power a trial to a given level (e.g., 80%) one must first estimate values for σ_{w}² and ρ_{w} using prior data and/or expert opinion. The power formula can then be composed with the variance formula with σ_{w}² and ρ_{w} fixed at their estimates \hat{σ}_{w}² and \hat{ρ}_{w}. The resulting function returns power for any values of n₀ and n₁.

The goal of a sample size calculation in the design of a clinical trial (e.g., that uses PROCOVA) can be to estimate n₀ and n₁ required to achieve the required power. However, one needs an additional constraint such as (but not limited to) a chosen randomization ratio n₀/n₁, and/or minimizing the total trial size n₀+n₁. In some examples, the randomization ratio may be pre-specified, but the same principles can be easily applied to other situations.

In many cases, a trial will aim to assess the effect of the intervention on many different outcomes. Processes in accordance with several embodiments of the invention can use multiple prognostic models (e.g., one to predict each outcome of interest) and/or multivariate prognostic models. Depending on the variances of the outcomes, and the accuracy with which they can be predicted, sample size calculations on the various outcomes of interest may suggest different required sample sizes. In this case, one could simply choose the smallest sample size that meets the minimum required statistical power on each of the outcomes of interest.

### B. Bayesian PROCOVA

In accordance with many embodiments of the invention, unbiased and precise treatment effect inferences may be obtained through: (1) adjusting for covariates that are highly correlated with the outcome, and (2) leveraging historical control information via Bayes' theorem. The Bayesian prognostic covariate adjustment methodology known as Bayesian PROCOVA can combine both objectives, incorporating digital twin generators (DTGs), that are trained on historical (e.g., control) data to produce digital twin (DT) probability distributions for individual participants' control outcomes. In doing so, expected values of the DT distributions may be leveraged to define covariates that will be adjusted. Additional implementations of Bayesian PROCOVA are disclosed in D. Walsh et al. (2020) “Bayesian prognostic covariate adjustment,” Unlearn.AI, Inc., Available at: https://arxiv.org/abs/2012.13112, incorporated by reference in its entirety.

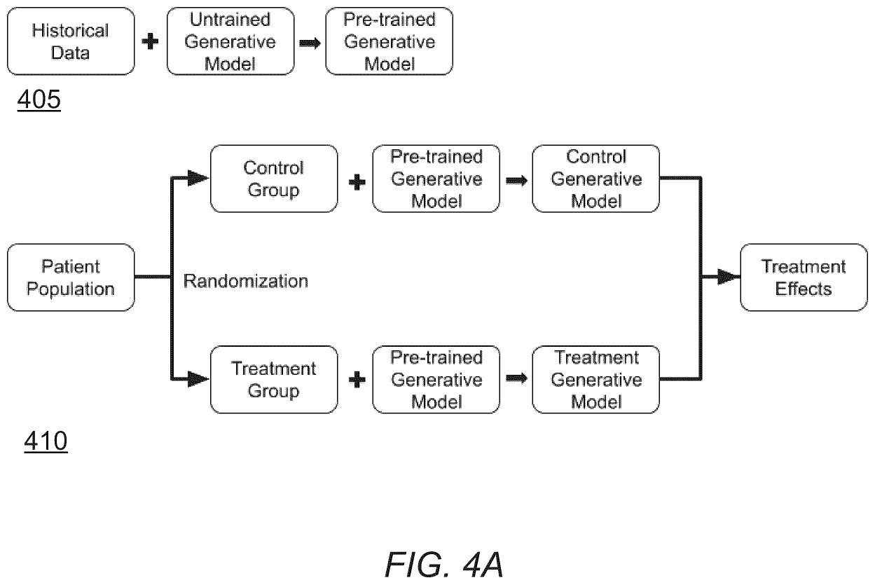

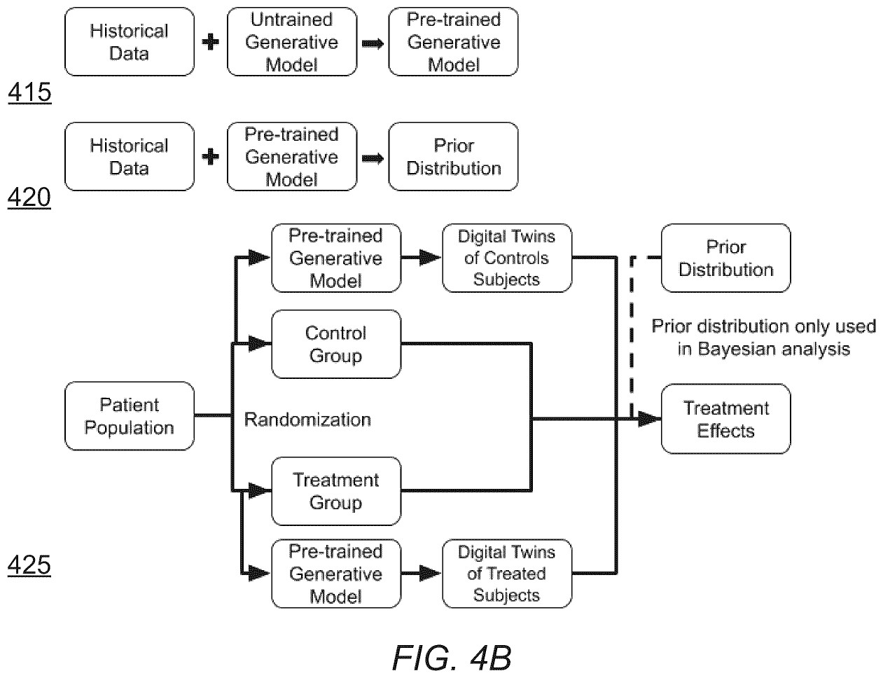

An example of using generative models to estimate treatment effects in accordance with multiple embodiment of the invention is illustrated in FIG. 4A. In the first part 405, an untrained generative model of the control condition is trained using historical data, such as (but not limited to), data from previously completed clinical trials, electronic health records, and/or other studies. In the second part 410, a patient population is randomly divided into a control group and a treatment group as part of a randomized controlled trial. Patients from the population can be randomized into the control and treatment groups with unequal randomization in accordance with a variety of embodiments of the invention. In this example, two new generative models are trained: one for the control group and one for the treatment group. In certain embodiments, control and treatment generative models can be based on a pre-trained generative model but can be additionally trained to reflect new information from the RCT. Outputs from the control and generative models (e.g., digital twins) can then be compared to compute the treatment effects. In several embodiments, Bayesian methods (and/or bootstrap) may be used to estimate uncertainties in the treatment effects and decision rules based on p-values and/or posterior probabilities may be applied.

An example of borrowing information from digital twins to estimate treatment effects in accordance with some embodiments of the invention is illustrated in FIG. 4B. In the first part 415, a generative model of the control condition is trained using historical data from previously completed clinical trials, electronic health records, or other studies. In the second part 420, when the analysis to be performed is Bayesian, predictions from the generative model are compared to historical data that were not used to train the model in order to obtain a prior distribution capturing how well the predictions generalize to new populations. In the third part 425, a randomized controlled trial is conducted (potentially with unequal randomization), digital twins are generated for each subject in the trial, and all of the data are incorporated into a statistical analysis (including the prior from part 420 when the analysis is Bayesian) to estimate the treatment effects. Bayesian methods and analytical calculations thereby be used to estimate uncertainties in the treatment effects (as disclosed further below).

### 1. General

Bayesian inference may refer to the fitting of statistical models to data so as to obtain probability distributions on the unknown model parameters θ. Under this paradigm, all uncertainties and information may be encoded via probability distributions, and all inferences and conclusions may be obtained via the laws of probability theory. The direct and explicit use of probability, specifically via the prior and posterior probability distributions for θ, for quantifying uncertainty and information for all unknown parameters is the essential characteristic of Bayesian inference.

The necessary elements for Bayesian inference may include but are not limited to the prior probability distribution for θ and the likelihood function for θ. The prior probability distribution, p(θ), may encode information about θ that is contained in historical data, and may augment the information from the RCT(s). Additionally or alternatively, the likelihood function may be denoted by L(θ|y, w, X), where y=(y₁, . . . , y_{N})^{T} is the vector of the participants' observed outcomes, w=(w₁, . . . , w_{N})^{T} is the vector of their treatment assignments, and

$$X = ( \begin{matrix}{x}_{1}^{T} \\ ⋮ \\ {x}_{N}^{T}\end{matrix} )$$

is the matrix of their covariates. The likelihood function may encode information about θ from the RCT(s), and be obtained from the sampling distribution of the data as specified by the generative statistical model underlying the analysis. The prior and likelihood functions may be combined via Bayes' theorem to calculate the posterior probability distribution p(θ|y, w, X) of θ conditional on the data. In accordance with several embodiments, the posterior distribution can be calculated as a proportional quantity, without a normalization constant, according to p(θ|y, w, X)≢p(θ)L(θ|y, w, X).

Bayesian PROCOVA may incorporate information including but not limited to (target) RCT data and/or data from historical control datasets, i.e., datasets independent of the RCT in which all participants are given control treatments. In this disclosure, causal estimands are defined as comparisons of potential outcomes for a set of experimental units. The experimental units could correspond to populations including but not limited to the finite-population of participants in the (target) RCT and/or to the conceptual “super-population” of all potential participants that could have been recruited into the (target) RCT. For example, the quantity Y̅(1)−Y̅(0)=N⁻¹Σᵢ₌₁^{N}{Yᵢ(1)−Yᵢ(0)} may be considered the finite-population average treatment effect. Additionally or alternatively, the quantity μ(1)−μ(0)=E{Y(1)}− E{Y(0)} may be considered the super-population average treatment effect. Systems implemented in accordance with some embodiments of the invention may denote E{Y(w)}=∫_{-∞}^{∞}ydF_{w}(y) as the expected value of the potential outcomes under treatment w∈{0, 1} for the super-population of participants as defined by the cumulative distribution function F_{w}: [symbol] →(0,1); his may also be referred to as the prognostic score. Moreover, as mentioned above, the historical control data may be used to specify the prior distribution(s) for the model parameters. Systems denote the sample size for the historical control data by N_{H}, the covariate vector for participant i=1, . . . , N_{H} by x_{i,H} ∈ [symbol] ^{L}, and their outcome by y_{i,H}.

Under the Neyman-Rubin Causal Model, validity of causal inferences, including but not limited to the likelihood function for Bayesian PROCOVA, may be heavily dependent on treatment assignment mechanisms; for example, the probability mass function p(w₁, . . . , w_{N}|Y₁(0), Y₁(1), . . . , Y_{N}(0) Y_{N}(1), x₁ . . . , x_{N}). Systems configured in accordance with several embodiments may therefore assume that treatment assignment mechanisms are probabilistic, individualistic, and unconfounded. These three assumptions may enable an “automated” specification of the likelihood function for Bayesian inference on causal estimands, in terms of the observed outcomes yᵢ=wᵢYᵢ(1)+(1−wᵢ)Y(0) in the RCTs.

In accordance with miscellaneous embodiments of the invention, posterior distributions p(θ|y, w, X) may encode all information about θ that is contained in the prior and/or RCT data. All inferences on causal estimands can be obtained from this distribution. To illustrate, consider the finite-population average treatment effect Y̅(1) −Y̅(0). Bayesian inference for this estimand requires the calculation of its posterior distribution, conditional on y, w, and X. Following the above reasoning the posterior distribution is calculated by using p(θ|y, w, X) to impute the missing potential outcomes yᵢ^{mis}=(1−wᵢ)Yᵢ(1)+wᵢYᵢ(0), calculating the estimand for each such imputation based on the observed and imputed missing outcomes according to N⁻¹Σᵢ₌₁^{N}{(2wᵢ−1)(yᵢ-yᵢ^{mis})}, and concatenating all such calculated estimands. More formally, the posterior distribution for Y̅(1)−Y̅(0) is calculated according to the integration p(Y̅(1)−Y̅(0)|y, w, X)=∫{p(Y̅(1)−Y̅(0)|y^{mis}, θ, y, w, X)p(y^{mis}|θ, y, w, X)p(θ|y, w, X)}dy^{mis}dθ, where y^{mis}=(y₁^{mis}, . . . y_{N}^{mis})^{T}. Given this posterior distribution, point estimates of the causal estimand can be obtained via mean(s), median(s), and/or mode(s), and interval estimates can be obtained by computing quantiles of the posterior distribution.

Bayesian inferences, implemented in accordance with certain embodiments of the invention for the super-population average treatment effect μ(1)−μ(0) may be obtained by the posterior distributions for specified parameters in the linear regression model used as the data generating mechanism (e.g., DTG):

$${Y}_{i} ( w ) = {v}_{i}^{T} β + {ϵ}_{i} ( w ) \qquad ( 1 )$$

where vᵢ ∈ [symbol] ^{K} is the vector of predictors for participant i=1, . . . , N that are defined as functions of wᵢ and xᵢ; β=(β₀, . . . , β_{K-1})^{T} is the vector of regression coefficients, and the ϵᵢ(w) are random error terms distributed according to [ϵᵢ(w)|X, β, σ²]˜N(0, σ²) with variance parameter σ². In accordance with multiple embodiments of the invention, the first two entries in each vᵢ are v_{i,1}=1 and v_{i,2}=wᵢ, and the β₁ entry in β corresponds to the super-population average treatment effect. As the observed outcomes are a function of the potential outcomes and treatment indicator, the data generating model in equation (1) may motivate the linear regression model for the observed outcomes according to:

$${y}_{i} = {v}_{i}^{T} β + {ϵ}_{i} \qquad ( 2 )$$

where the [ϵᵢ|X, β, σ²]˜N(0, σ²) independently as before.

In accordance with multiple embodiments of the invention, Bayesian inferences may be performed on parameters including but not limited to β₁ by extending the linear regression model (2) with a prior distribution on β, σ² and calculating the posterior distribution for all parameters according to Bayes' theorem. The unconfoundedness assumption may thereby lead to the derivation of the likelihood function as:

$$L ( β , {σ}^{2} \vert y , w , X ) = {( {σ}^{2} )}^{- N / 2} exp \{ - \frac{1}{2 {σ}^{2}} {( y - V β )}^{T} ( y - V β ) \} \qquad ( 3 )$$

where

$$V = ( \begin{matrix}{v}_{1}^{T} \\ ⋮ \\ {v}_{N}^{T}\end{matrix} ) .$$

Meanwhile, the posterior distribution can be calculated as:

$$p ( β , {σ}^{2} \vert y , w , X ) ∝ p ( β , {σ}^{2} ) {( {σ}^{2} )}^{- \frac{N}{2}} exp \{ - \frac{1}{2 {σ}^{2}} {( y - V β )}^{T} ( y - V β ) \} . \qquad ( 4 )$$

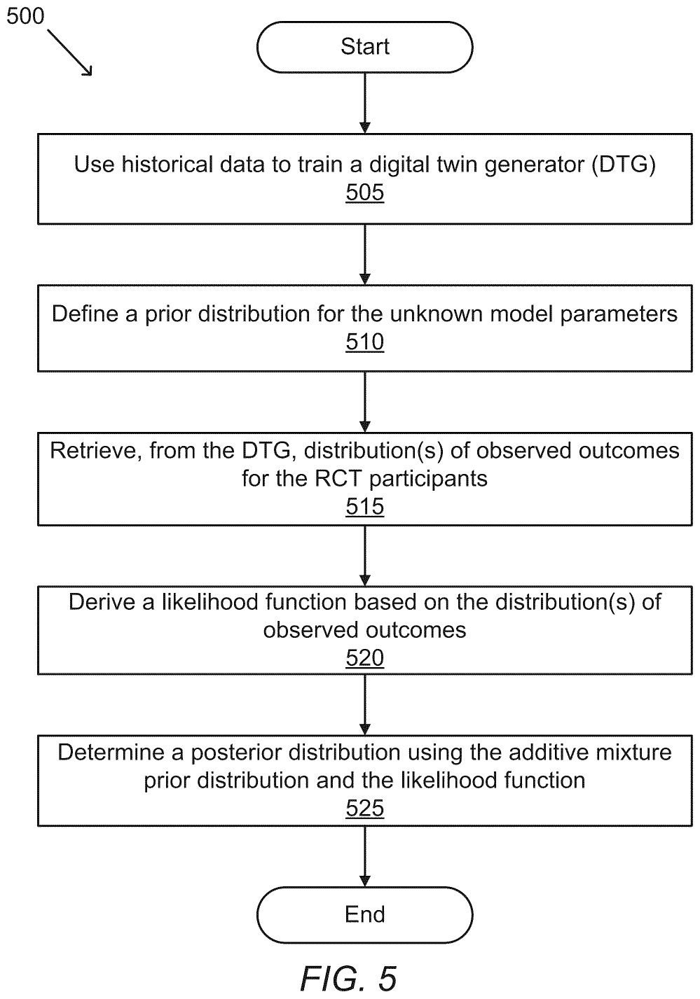

An example of the Bayesian PROCOVA process applied in accordance with multiple embodiments of the invention is illustrated in FIG. 5. Process 500 uses (505) historical data to train a digital twin generator (DTG) and defines a prior distribution. In accordance with many embodiments of the invention, historical control data may include but is not limited to control arm data from historical control arms of previous RCT trials and/or previous digital twin outcomes. In some embodiments of the invention, training (505) DTGs may not involve any target RCT (e.g., treatment) data. In doing so, the DTGs may be completely prespecified.

Process 500 defines (510) a prior distribution for the unknown model (i.e., DTG) parameters based, at least in part, on the historical control data. As suggested above, in accordance with many embodiments of the invention, the prior distribution may be a joint distribution for the unknown parameters which may include but are not limited to ƒ (the regression coefficients) and σ² (the outcome variance). Further, the form the prior distribution takes may vary. As discussed in the next section, in accordance with many embodiments of the invention, an additive mixture prior may be used that corresponds to the combination of an informative prior (based on the historical control data) and a non-informative prior (not based on the historical control data) distribution according to a weight parameter. Additionally or alternatively, in accordance with various embodiments of the invention (as discussed in the “Bayesian PROCOVA v.2” section) a discounted mixture prior distribution may be used that partially discounts the historical control data (when sufficiently large).

Process 500 retrieves (515), from the DTG, distribution(s) of observed outcomes for the RCT participants. In many embodiments, the observed outcomes may represent the potential results under treatment w∈{0, 1} for the super-population of participants, if they were assigned to the control group of a given RCT. In accordance with some embodiments of the invention, the (e.g., sole) inputs for trained DTGs may be participant covariates and baseline measurements. As such, they may avoid introducing any bias in treatment effect inferences for the RCT. Probability distributions generated in accordance with certain embodiments of the invention may consider each participant's fixed timepoint post-treatment assignment. As referenced above in relation to causal estimands, process 500 may represent the observed outcomes by the cumulative distribution function (CDF) F_{w}: [symbol] →(0, 1) and/or a corresponding prognostic score [symbol] {Y(w)}=∫_{-∞}^{∞}ydF_{w}(y) for the associated RCT participant. In accordance with various embodiments of the invention, prognostic scores may be calculated via Monte Carlo samples (i.e., by obtaining independent samples from each distribution F_{w} and calculating the averages of the respective samples).

Process 500 derives (520) a likelihood function based on the distribution(s) of observed outcomes. In accordance with certain embodiments of the invention, likelihood functions for Bayesian PROCOVA may be derived as a consequence of the generative statistical model (e.g., DTG) outputs. Systems configured in accordance with many embodiments of the invention may denote the likelihood function by: L(θ|y, X), where y=(y₁, . . . , y_{N})^{T} and

$$X = ( \begin{matrix}1 & {w}_{1} & ( {m}_{1} - \overline{m} ) \\ 1 & {w}_{2} & ( {m}_{2} - \overline{m} ) \\ ⋮ & ⋮ & ⋮ \\ 1 & {w}_{N} & ( {m}_{N} - \overline{m} )\end{matrix} ) .$$

The likelihood function may therefore take the form:

$$L ( θ \vert y , X ) = {( {σ}^{2} )}^{- \frac{N}{2}} exp \{ - \frac{1}{2 {σ}^{2}} {( y - \overline{m} 1 - X β )}^{T} ( y - \overline{m} 1 - X β ) \} \qquad ( 5 A )$$

where 1 is the N×1 vector in which each entry is 1, and β=(β₀, β_{w}, βₘ)^{T}. Systems may, additionally or alternatively, define the centered outcomes yᵢ^{(c)}=yᵢ−m̅ and the vector y^{(c)}=(y₁^{(c)}, . . . y_{N}^{(c)})^{T} in the RCT to simplify the expression for the likelihood as:

$$L ( θ \vert y , X ) = {( {σ}^{2} )}^{- \frac{N}{2}} exp \{ - \frac{1}{2 {σ}^{2}} {( {y}^{( c )} - X β )}^{T} ( {y}^{( c )} - X β ) \} . \qquad ( 5 B )$$

Process 500 determines (525) a posterior distribution using the prior distribution and the likelihood function. In accordance with numerous embodiments, the determined posterior distribution may be used for various applications related to target RCTs, as will be discussed below. Potential applications of the posterior distribution may include but are not limited to: deriving posterior means and/or variances to determine values for uncertainty/bias, updating system weights (e.g., weight parameter prior distributions), evaluating treatment effects, deriving a variance reduction, and/or performing a control arm sample size reduction.

While specific processes for Bayesian PROCOVA are described above in FIG. 5, any of a variety of processes can be utilized to evaluate unknown trial parameters as appropriate to the requirements of specific applications. In some embodiments, steps may be executed or performed in any order or sequence not limited to the order and sequence shown and described. In numerous embodiments, some of the above steps may be executed or performed substantially simultaneously where appropriate or in parallel to reduce latency and processing times. In a number of embodiments, one or more of the above steps may be omitted.

### 2. Bayesian PROCOVA v.1—Additive Mixture Prior

In accordance with many embodiments of the invention, historical control information can be leveraged via an (additive mixture) prior that is completely pre-specifiable, and therefore does not involve any information from soon-to-be implemented RCTs. Additive mixture priors may be implemented with two components: an informative prior probability distribution specified based on historical control data, and a non-informative prior distribution. Moreover, the respective prior distributions (i.e., informative and non-informative) may be combined, following the aforementioned additive mixture, according to at least one weight parameter. The weight parameter(s) in the mixture may themselves follow a prior distribution. Additionally or alternatively, in accordance with several embodiments of the invention, multiplicative mixture priors (e.g., with fixed weights) may be implemented when performing Bayesian PROCOVA. Nevertheless, this disclosure will primarily refer to the additive mixture prior implementations.

Prior configurations implemented in accordance with various embodiments of the invention may enable resulting posterior distributions to dynamically borrow information from historical control data, thereby effectively augmenting RCT information. In scenarios where historical control data and RCT data are consistent with one another, Bayesian PROCOVA may be configured to put significant weight on the information from the historical control data, consequently increasing the precision of treatment effect inferences from the RCT. When historical control and RCT data are discrepant, Bayesian PROCOVA may be configured to discount the historical control data and, in doing so, yield inferences similar to PROCOVA in terms of controlling bias in treatment effect inferences.

In accordance with numerous embodiments of the invention, implementations of Bayesian PROCOVA may correspond to a Bayesian linear regression model. This Bayesian linear regression model may, in part, be based on using: an informative prior distribution p₁(θ), defined based on historical control data, as one component of the prior distribution p(θ) for θ; and a non-informative (colloquially known as “flat”) prior distribution p_{F}(θ). Furthermore, the use of dynamic information borrowing may account for concerns about biased inferences (e.g., where historical control data is discrepant with RCTs), balancing the objectives of controlling bias in inferences while increasing the precision of the inferences. As mentioned above, where the historical data is consistent with an RCT, then significant weight may be placed on p₁(θ) to define p(θ). Additionally or alternatively, where historical data is inconsistent with the RCT then significant weight is placed on p_{F}(θ) to define p(θ).

Systems configured in accordance with several embodiments of the invention may adopt the following generative statistical (i.e., mean) model for Bayesian PROCOVA:

$${y}_{i} = {β}_{0} + {β}_{w} {w}_{i} + {β}_{m} ( {m}_{i} - \overline{m} ) + \overline{m} + {ϵ}_{i} \qquad ( 6 )$$

where yᵢ ∈ [symbol] is the endpoint for participant i=1, . . . , N in the RCT; wᵢ ∈{0,1} is the participant's treatment indicator (with 0 denoting control and 1 denoting active treatment); mᵢ ∈ [symbol] is their prognostic score;

$$\overline{m} = \frac{1}{N} {\sum }_{i = 1}^{N} {m}_{i} ;$$

the noise [ϵᵢ|w, m, β₀, β_{w}, βₘ, σ²]˜N(0, σ²) independently for w=(w₁, . . . , w_{N})^{T} and m (m₁, . . . m_{N})^{T}; and the unknown parameters are θ=(β0, β_{w}, βₘ, σ²)^{T}. Implementations in accordance with various embodiments of the invention may therefore follow multiple assumptions. A first assumption may be that the mᵢ are independent and identically distributed random variables with finite mean, and their probability distribution does not depend on θ. A second assumption may be that the joint probability mass function of w is not a function of m or the unknown parameters, so that ƒ(w|m, θ)=ƒ(w) in terms of the joint probability mass function. A third assumption may be that fixed designs are used throughout, i.e., N₁ participants will be assigned treatment and N−N₁ participants will be assigned control in any RCT where N₁=π₁N, where π₁ represents the proportion of RCT participants in the treatment group. More formally, in accordance with some embodiments of the invention, fixed designs may follow the configuration:

$$f ( w ) = \{ \begin{matrix}{( \begin{matrix}N \\ {N}_{1}\end{matrix} )}^{- 1} & if {\sum }_{i = 1}^{N} {w}_{i} = {N}_{1} \\ 0 & otherwise\end{matrix} . \qquad ( 6 )$$

As suggested in the previous section, under this formulation and set of assumptions, β₀ may interpreted as the bias of the average of the prognostic scores in predicting the endpoint of a participant in the control group, i.e., β₀= [symbol] (yᵢ−m̅ |wᵢ=0, θ).

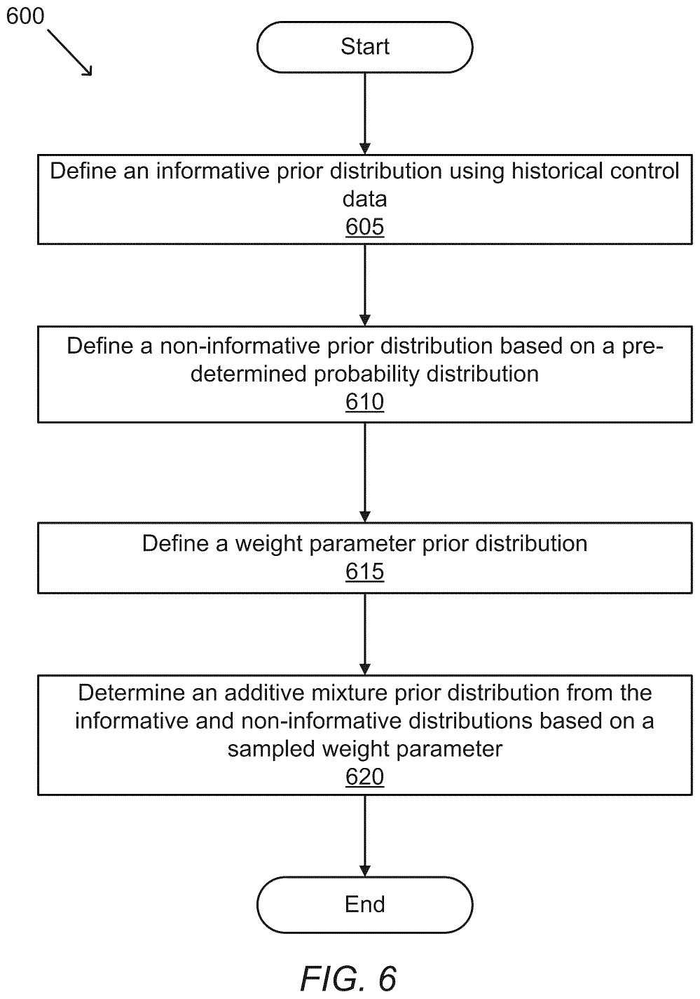

A process for determining an additive mixture prior in accordance with some embodiments of the invention is illustrated in FIG. 6. Process 600 defines (605) an informative prior distribution using historical control data. In accordance with several embodiments of the invention, informative components (p₁(θ)) of additive mixture priors can be defined as a combination of two probability distributions:

$$[ {β}_{0} , {β}_{w} , {β}_{m} \vert {σ}^{2} ] ∼ N ( ( \begin{matrix}{\hat{β}}_{0 , H} \\ 0 \\ {\hat{β}}_{m , H}\end{matrix} ) , ( \begin{matrix}{σ}^{2} {K}_{0 , H} & 0 & 0 \\ 0 & {σ}^{2} {K}_{w} & 0 \\ 0 & 0 & {σ}^{2} {K}_{m , H}\end{matrix} ) )$$

$${σ}^{2} ∼ \frac{( L - 2 ) {s}_{H}^{2}}{{χ}_{L - 2}^{2}}$$

where the distributions are defined via the following hyperparameters: \hat{β}_{0,H} and \hat{β}_{m,H} are point estimates of β₀ and βₘ (respectively) based on historical control data (both digital twins and outcomes); K_{0,H}, K_{w}, and K_{m,H} represent proportionality constants governing the prior variances of β₀, β_{w}, and βₘ (respectively), with K_{0,H}, K_{m,H} based on historical control data; L−2 represents the prior degrees of freedom used for estimating σ² from a historical control dataset involving L participants; and s_{H}² is the point estimate of σ² from the historical control data.

Process 600 defines (610) a non-informative prior distribution based on a pre-determined probability distribution. In many implementations, systems operating in accordance with certain embodiments may consider additive mixture priors where the weight (ω) is a random variable with a specified weight parameter prior distribution (p(ω)). As 0<ω<1, classes of probability distributions that can be used for the weight parameter prior distribution may include but are not limited to the Beta distribution. In accordance with various embodiments of the invention, the probability density function for a Beta(α₁, α₂) random variable may take the form:

$$p ( ω ) = \frac{Γ ( {α}_{1} + {α}_{2} )}{Γ ( {α}_{1} ) Γ ( {α}_{2} )} {{ω}^{{α}_{1} - 1} ( 1 - ω )}^{{α}_{2} - 1} \qquad ( 7 )$$

where α₁, α₂>0. Additionally or alternatively, the non-informative (flat) prior distribution (p_{F}(θ)) may be defined via the combination of the following two probability distributions:

$$[ {β}_{0} , {β}_{w} , {β}_{m} \vert {σ}^{2} ] ∼ N ( ( \begin{matrix}0 \\ 0 \\ 0\end{matrix} ) , {σ}^{2} {KI}_{N \times N} )$$

$${σ}^{2} ∼ \frac{{v}_{0} {σ}_{0}^{2}}{{χ}_{{v}_{0}}^{2}} .$$

where, of the hyperparameters: K represents a proportionality constant for the prior variances of β₀, β_{w}, βₘ; v₀ represents the prior degrees of freedom for σ²; and σ₀² represents a prior point estimate of σ². In some cases, the prior degrees of freedom v₀ may be chosen to be small and/or the prior point estimate σ₀² may be chosen such that a resulting Inverse Chi-Squared probability distribution for the prior of σ² resembles the shape of the standard non-informative prior p(θ)≢(σ²)⁻¹. For example, one such pair of hyperparameter values is v₀=1 and σ₀²=1. Furthermore, as v₀→∞ and σ₀² is held fixed, priors implemented in accordance with many embodiments of the invention may converge to the standard non-informative prior.

Process 600 defines (615) a weight parameter prior distribution. Additive mixture priors implemented in accordance with various embodiments of the invention may combine multiple component probability distributions according to weight parameter(s) that follow a prior distribution (e.g., to emphasize one component over another). Systems operating in accordance with certain embodiments may consider additive mixture priors where the weight (ω) is a random variable with weight parameter prior distribution (p(ω)). As 0<ω<1, classes of prior distributions that can be used for the weight parameter prior distribution may include but are not limited to the Beta distribution. In accordance with various embodiments of the invention, the probability density function for a Beta(α₁, α₂) random variable may take the form:

$$p ( ω ) = \frac{Γ ( {α}_{1} + {α}_{2} )}{Γ ( {α}_{1} ) Γ ( {α}_{2} )} {{ω}^{{α}_{1} - 1} ( 1 - ω )}^{{α}_{2} - 1} \qquad ( 8 )$$

where α₁, α₂>0.

Process 600 determines (620) an additive mixture prior distribution from the informative and non-informative distributions based on a sampled weight parameter. In accordance with many embodiments of the invention, the general structure of additive mixture priors conditional on weight parameters may follow the formula:

$$p ( θ \vert ω ) = ω {p}_{I} ( θ ) + ( 1 - ω ) {p}_{F} ( θ ) \qquad ( 9 )$$

where 0<ω<1. Therefore when ω=0, and specifying p^{F}(θ)≢(σ²)⁻¹, Bayesian PROCOVA may effectively correspond to standard PROCOVA (excluding HC1 standard errors).

While specific processes for defining prior distributions are described above in FIG. 6, any of a variety of processes can be utilized to implement randomized controlled trials as appropriate to the requirements of specific applications. In some embodiments, steps may be executed or performed in any order or sequence not limited to the order and sequence shown and described. In several embodiments, some of the above steps may be executed or performed substantially simultaneously where appropriate or in parallel to reduce latency and processing times. In numerous embodiments, one or more of the above steps may be omitted.

### a) Weight Parameter for Prior Distribution

As mentioned above, in accordance with some embodiments of the invention, prior distributions may be established for the weight parameter. For example, prior density functions for weight parameters may be denoted by p(ω), while the joint prior density function for all the parameters in the model can take the form:

$$p ( ω , θ ) = p ( ω ) p ( θ \vert ω ) = ω p ( ω ) {p}_{I} ( θ ) + ( 1 - ω ) p ( ω ) {p}_{F} ( θ ) .$$

Moreover, in accordance with numerous embodiments of the invention, the amount of information provided by prior distributions p(θ|ω) of θ conditional on a fixed value of ω may vary. This question may be addressed by characterizing the prior information contained in p(θ|ω) in terms of an equivalent amount of information provided by a hypothetical RCT of a specified sample size. The sample size for the hypothetical RCT is referred to as the “effective prior sample size”.

Specifically, systems in accordance with certain embodiments of the invention may propose to quantify the prior information under Bayesian PROCOVA by comparing the prior variance of specified parameters in θ (e.g., β₀, β_{w}) to the posterior variance of those parameters (in a scenario where the RCT data were analyzed via a Bayesian linear regression model whose only predictor is the treatment indicator and whose prior is the standard flat prior). By setting the prior variance of a parameter (such as β_{w}) equal to the posterior variance of the same parameter (in the case of a flat prior and the Bayesian unadjusted analysis), systems can leverage closed-form expressions for the posterior variance to find the sample size for the hypothetical RCT such that the amount of information provided by the RCT on β would be equivalent to the amount of information on this parameter contained in p(θ|ω). Systems in accordance with a number of embodiments may consider the case of fixed weights for interpreting the prior effective sample size as they can be used for both trial planning purposes and for sensitivity analyses.

To illustrate this approach, β_{w} can be evaluated. In accordance with many embodiments of the invention, Var(μ_{w}|ω) can be calculated under additive (and/or multiplicative) mixture priors. For a hypothetical RCT with N₁ treated participants and N−N₁=N₀ control participants in which the outcomes are analyzed using a Bayesian linear regression model whose sole predictor is wᵢ and in which a flat prior is placed on (β₀, β_{w}, σ²)^{T}, the posterior variance of β_{w} would be s²(N₁⁻¹+N₀⁻¹), where s² is the estimate of σ² from the regression model. In cases of 1:1 designs, so that N₀=N₁=N/2, then systems implemented in accordance with multiple embodiments of the invention may set: Var(β_{w}|ω)=4s²N⁻¹ and solve for N to obtain:

$$N = \frac{4 {s}^{2}}{Var ( {β}_{w} \vert ω )} \qquad ( 10 )$$

An important requirement for this calculation is that Var(β_{w}|ω) exists, which can be assured when a proper, generative prior is specified with finite first and second moments. The same approach can be implemented for β in a straightforward manner as for β_{w}.

### b) Informative Component for Prior Distribution

In accordance with several embodiments of the invention, informative components of additive mixture priors can be specified via the combination of two probability distributions:

$$[ {β}_{0} , {β}_{w} , {β}_{m} \vert {σ}^{2} ] ∼ N ( ( \begin{matrix}{\hat{β}}_{0 , H} \\ 0 \\ {\hat{β}}_{m , H}\end{matrix} ) , ( \begin{matrix}{σ}^{2} {K}_{0 , H} & 0 & 0 \\ 0 & {σ}^{2} {K}_{w} & 0 \\ 0 & 0 & {σ}^{2} {K}_{m , H}\end{matrix} ) )$$

$${σ}^{2} ∼ \frac{( L - 2 ) {s}_{H}^{2}}{{χ}_{L - 2}^{2}} .$$

This form for the informative component carries several benefits. First, the propriety of this component (in terms of being a proper probability distribution) helps establish that the posterior distribution in general is proper. Moreover, the joint prior probability distribution p(β₀, βₘ, σ²) corresponds to the posterior distribution of these parameters obtained by fitting the Bayesian linear regression model to the historical data under the standard non-informative prior. Additionally, selecting and justifying values for the hyperparameters above is conceptually straightforward. Systems implemented in accordance with several embodiments of the invention can validate Monte Carlo samplers for calculating the posterior distribution under the mixture prior (because the informative component is generative). Finally, the above form means that the calculation of the posterior distribution under the mixture prior can be simplified because the informative component is conjugate to the likelihood function.

In many embodiments of the invention, the marginal distribution of y may take the form of an N-dimensional Multivariate t-distribution with L−2 degrees of freedom, with the center equal to:

$$X ( \begin{matrix}{\hat{β}}_{0 , H} \\ 0 \\ {\hat{β}}_{m , H}\end{matrix} ) + \overline{m} 1$$

and scale matrix equal to:

$${s}_{H}^{2} ( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{T} ) .$$

In accordance with some embodiments of the invention, The informative component for the mixture prior may use the following joint probability density function:

$${p}_{I} ( θ ) ∝ {( {σ}^{2} )}^{- \{ ( L + 1 ) / 2 + 1 \}} exp [ - \frac{( L - 2 ) {s}_{H}^{2}}{2 {σ}^{2}} - \frac{1}{2 {σ}^{2}} \{ \frac{{( {β}_{0} - {\hat{β}}_{0 , H} )}^{2}}{{K}_{0 , H}} + \frac{{β}_{w}^{2}}{{K}_{w}} + \frac{{( {β}_{m} - {\hat{β}}_{m , H} )}^{2}}{{K}_{m , H}} \} ]$$

where the values of \hat{β}_{0,H}, β_{m,H}, K_{0,H}, K_{m,H}, s_{H}² are set by fitting a standard linear regression model to historical control data. Specifically, let:

$${X}_{H} = ( \begin{matrix}1 & ( {m}_{1 , H} - {\overline{m}}_{H} ) \\ 1 & ( {m}_{2 , H} - {\overline{m}}_{H} ) \\ ⋮ & ⋮ \\ 1 & ( {m}_{L , H} - {\overline{m}}_{H} )\end{matrix} ) \qquad ( 11 )$$

where m_{l,H} denotes the prognostic score for participant l=1, . . . , L in the historical control dataset and

$${\overline{m}}_{H} = \frac{1}{L} {\sum }_{l = 1}^{L} {m}_{l , H} .$$

Additionally or alternatively, y_{H}^{(c)}=(y_{1,H}^{(c)}, . . . , y_{L,H}^{(c)})^{T} may denote the vector of centered endpoints in the historical control dataset. Then:

$$( \begin{matrix}{\hat{β}}_{0 , H} \\ {\hat{β}}_{m , H}\end{matrix} ) = {( {X}_{H}^{T} {X}_{H} )}^{- 1} {X}_{H}^{T} {y}_{H}^{( c )} \qquad ( 12 )$$

where K_{0,H} and K_{m,H} are the (1,1) and (2,2) entries of (x_{H}^{T}x_{H})⁻¹ (respectively), and

$${s}_{H}^{2} = \frac{1}{L - 2} {\sum }_{l = 1}^{L} \{ {y}_{l , H}^{( c )} - {\hat{β}}_{0 , H} - {\hat{β}}_{m , H} ( {m}_{i , H} - {\overline{m}}_{H} ) \} .$$

By direct calculation, systems implemented in accordance with a few embodiments of the invention may have

$${K}_{0 , H} = \frac{1}{{N}_{H}} and / or {K}_{m , H} = \frac{1}{{\sum }_{i = 1}^{n} {( {m}_{i , H} - {\overline{m}}_{H} )}^{2}} .$$

Nevertheless, in practice, setting

$${K}_{0 , H} = \frac{1}{{N}_{H}}$$

can lead to inferences that are too overconfident. This is especially a concern in the case of domain shift. Thus, another specification that systems implemented in accordance with multiple embodiments of the invention may consider is to deflate K_{0,H} as a function of the historical control data sample size (e.g.,

$${K}_{0 , H} = \frac{1}{\sqrt{{N}_{H}}} ) .$$

In accordance with various embodiments, the value of K_{w} (the proportionality constant that governs the prior variance for β_{w}) may be set to a large number to make the prior variance for the treatment effect large, thereby making the prior for the treatment effect “weakly informative.” The specific value for this hyperparameter may depend on the specific endpoint under consideration. In practice, systems implemented in accordance with some embodiments of the invention can take K_{w}→∞ and consider a flat prior for μ_{w} in the informative component. Furthermore, systems may implement K_{m,H}→∞ and consider a flat prior for βₘ. Additionally or alternatively, in accordance with certain embodiments of the invention, the informative component of the additive mixture prior may be fully generative.

### c) Flat (Non-Informative) Component for Prior Distribution

As above, the flat component of the prior may be specified via the combination of two probability distributions:

$$[ {β}_{0} , {β}_{w} , {β}_{m} \vert {σ}^{2} ] ∼ N ( ( \begin{matrix}0 \\ 0 \\ 0\end{matrix} ) , {σ}^{2} {KI}_{N \times N} )$$

$${σ}^{2} ∼ \frac{{v}_{0} {σ}_{0}^{2}}{{χ}_{{v}_{0}}^{2}} .$$

Implementation of such a form for the flat component may provide various benefits. First, the propriety of this component (in terms of it being a proper probability distribution) helps to establish that the posterior distribution is proper. Additionally, various techniques may be used to validate Monte Carlo samplers for calculating the posterior distribution under the mixture prior (because the flat component is generative). Finally, calculation of the posterior distribution under the additive mixture prior may simplified because the flat component is conjugate to the likelihood function.

Under this flat component prior configuration, the marginal distribution of y may be an N-dimensional Multivariate t distribution with v₀ degrees of freedom, center at m̅1, and scale matrix equal to σ₀²(I_{N×N}+KXX^{T}).

In accordance with many embodiments of the invention, the flat component for the additive mixture prior may have the following joint probability density function:

$${p}_{F} ( θ ) ∝ {( {σ}^{2} )}^{- \{ ( {v}_{0} + 3 ) / 2 + 1 \}} exp \{ \frac{- {v}_{0} {σ}_{0}^{2}}{2 {σ}^{2}} - \frac{1}{2 K {σ}^{2}} ( {β}_{0}^{2} + {β}_{w}^{2} + {β}_{m}^{2} ) \}$$

where the selection of K depends on the scale of the endpoint, and the ideal value should correspond to a large number with respect to the scale of the endpoint. In some cases, the prior degrees of freedom v₀ may be chosen to be small and/or the prior point estimate α₀² may be chosen such that the resulting Inverse Chi-Squared probability distribution for the prior of σ² resembles the shape of the standard non-informative prior p(θ)≢(σ²)⁻¹. For example, one such pair of hyperparameter values is v₀=1 and σ₀²=1. Furthermore, as v₀→∞ and σ₀² is held fixed, priors implemented in accordance with many embodiments of the invention may converge to the standard non-informative prior. Additionally or alternatively, as prior p(θ)≢(σ²)⁻¹ is a limiting case of the proper prior, systems can take this limiting case and set the flat component equal to a standard non-informative prior distribution. Moreover, in accordance with many embodiments of the invention, the flat component of additive mixture prior may be fully generative.

### d) Posterior Distribution

As mentioned above, in accordance with a number of embodiments of the invention, posterior distributions can be calculated according to the formula:

$$p ( θ \vert ω , y , X ) ∝ L ( θ \vert ω , y , X ) p ( θ \vert ω ) .$$

Moreover, this implementation may be reconfigured by specifying a Multivariate Normal distribution for the conditional prior [β₀, β_{w}, βₘ|σ²] and/or an Inverse Chi-Squared distribution for the marginal prior of σ² under either or both of the informative and the flat components. Therefore, in accordance with many embodiments of the invention, for the informative and/or flat components, the conditional posterior [β₀, β_{w}, βₘ|σ², y, X] may follow a Multivariate Normal distribution, while the marginal posterior [σ²|y, X] may follow an Inverse Chi-Squared distribution. As such, the full posterior distribution may take the form: p(θ|ω, y, X)≢ωL(θ|ω, y, X)pₗ(θ)+(1−ω)L(θ|ω, y, X)p_{F}(θ).

In accordance with several embodiments of the invention, the normalization constant (C) for the posterior distribution may be calculated according to the expression:

$$C = {\{ ω \int L ( θ \vert ω , y , X ) {p}_{I} ( θ ) d θ + ( 1 - ω ) \int L ( θ \vert ω , y , X ) {p}_{F} ( θ ) d θ \}}^{- 1} \qquad ( 13 )$$

Given the marginal distributions under the informative and flat components, respectively, the closed-form expression for C⁻¹ is:

$${C}^{- 1} = \{ \frac{ω Γ ( \frac{N + L - 2}{2} )}{Γ ( \frac{L - 2}{2} ) {( L - 2 )}^{\frac{N}{2}} {π}^{\frac{N}{2}}} \} {( {s}_{H}^{2} )}^{- \frac{N}{2}} {\{ det ( {I}_{N \times N} + X ( \begin{matrix}{K}_{0 , H} & 0 & 0 \\ 0 & {K}_{w} & 0 \\ 0 & 0 & {K}_{m H}\end{matrix} ) {X}^{⊤} ) \}}^{- \frac{1}{2}} \times {\{ 1 + \frac{1}{( L - 2 ) {s}_{H}^{2}} {( {y}^{( c )} - X ( \begin{matrix}{\hat{β}}_{0 , H} \\ 0 \\ {\hat{β}}_{m , H}\end{matrix} ) )}^{⊤} {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1} ( {y}^{( c )} - X ( \begin{matrix}{\hat{β}}_{0 , H} \\ 0 \\ {\hat{β}}_{m , H}\end{matrix} ) ) \}}^{- ( \frac{N + L - 2}{2} )} + \{ \frac{( 1 - ω ) Γ ( \frac{N + {v}_{0}}{2} )}{Γ ( \frac{{v}_{0}}{2} ) {( {v}_{0} )}^{N / 2} {π}^{N / 2}} \} {( {σ}_{0}^{2} )}^{- N / 2} {\{ det ( {I}_{N \times N} + K X {X}^{⊤} ) \}}^{- 1 / 2} \times {\{ 1 + ( \frac{1}{{v}_{0} {σ}_{0}^{2}} ) {( {y}^{( c )} )}^{⊤} {( {I}_{N \times N} + K X {X}^{⊤} )}^{- 1} {y}^{( c )} \}}^{- ( \frac{N + {v}_{0}}{2} )} .$$

Accounting for the above, weights for the two components of the posterior distribution may take the form C^{ω}∫L(θ|ω, y, X)pₗ(θ)dθ (for the informative component) and C(1−ω)∫L(θ|ω, y, X)p_{F}(θ)dθ (for the flat component). The closed-form expression for the weight of the first component may take the form:

$${ω}_{*} = \{ \frac{C ω Γ ( \frac{N + L - 2}{2} )}{Γ ( \frac{L - 2}{2} ) {( L - 2 )}^{N / 2} {π}^{N / 2}} \} {( {s}_{H}^{2} )}^{- N / 2} \{ det {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1 / 2} \times {\{ 1 + \frac{1}{( L - 2 ) {s}_{H}^{2}} {( {y}^{( c )} - X ( \begin{matrix}{\hat{β}}_{0 , H} \\ 0 \\ {\hat{β}}_{m , H}\end{matrix} ) )}^{⊤} {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1} ( {y}^{( c )} - X ( \begin{matrix}{\hat{β}}_{0 , H} \\ 0 \\ {\hat{β}}_{m , H}\end{matrix} ) ) \}}^{- ( \frac{N + L - 2}{2} )}$$

and the closed-form expression for the weight of the second component may take the form:

$$1 - {ω}_{*} = \{ \frac{C ( 1 - ω ) Γ ( \frac{N + {v}_{0}}{2} )}{Γ ( \frac{{v}_{0}}{2} ) {( {v}_{0} )}^{N / 2} {π}^{N / 2}} \} {( {σ}_{0}^{2} )}^{- N / 2} {\{ det ( {I}_{N \times N} + K X {X}^{⊤} ) \}}^{- 1 / 2} \times {\{ 1 + ( \frac{1}{{v}_{0} {σ}_{0}^{2}} ) {( {y}^{( c )} )}^{⊤} {( {I}_{N \times N} + K X {X}^{⊤} )}^{- 1} {y}^{( c )} \}}^{- ( \frac{N + {v}_{0}}{2} )} .$$

In accordance with a number of embodiments of the invention, posterior distributions can be calculated according to the formula:

$$p ( θ \vert ω , y , X ) ∝ L ( θ \vert ω , y , X ) p ( θ \vert ω ) .$$

Moreover, this implementation may be reconfigured by specifying a Multivariate Normal distribution for the conditional prior [β₀, β_{w}, βₘ|σ²] and/or an Inverse Chi-Squared distribution for the marginal prior of σ² under both the informative and the flat components. Therefore, in accordance with many embodiments of the invention, for the informative and/or flat components, the conditional posterior [β₀, β_{w}, βₘ|σ², y, X] may follow a Multivariate Normal distribution, while the marginal posterior [σ²|y, X] may follow an Inverse Chi-Squared distribution. As such, the full posterior distribution may take the form:

$$p ( θ \vert ω , y , X ) ∝ ω L ( θ \vert ω , y , X ) {p}_{I} ( θ ) + ( 1 - ω ) L ( θ \vert ω , y , X ) {p}_{F} ( θ ) .$$

In accordance with several embodiments of the invention, the normalization constant (C) for the posterior distribution may be calculated according to:

$$C = {\{ ω \int L ( θ \vert ω , y , X ) {p}_{I} ( θ ) d θ + ( 1 - ω ) \int L ( θ \vert ω , y , X ) {p}_{F} ( θ ) d θ \}}^{- 1} \qquad ( 14 )$$

Given the marginal distributions under the informative and flat components, respectively, the closed-form expression for C⁻¹ may take the form:

$${C}^{- 1} = \{ \frac{ω Γ ( \frac{N + L - 2}{2} )}{Γ ( \frac{L - 2}{2} ) {( L - 2 )}^{N / 2} {π}^{N / 2}} \} {( {s}_{H}^{2} )}^{- N / 2} {\{ det ( {I}_{N \times N} + X ( \begin{matrix}{K}_{0 , H} & 0 & 0 \\ 0 & {K}_{w} & 0 \\ 0 & 0 & {K}_{m H}\end{matrix} ) {X}^{⊤} ) \}}^{- 1 / 2} \times {\{ 1 + \frac{1}{( L - 2 ) {s}_{H}^{2}} {( {y}^{( c )} - X ( \begin{matrix}{\hat{β}}_{0 , H} \\ 0 \\ {\hat{β}}_{m , H}\end{matrix} ) )}^{⊤} {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1} ( {y}^{( c )} - X ( \begin{matrix}{\hat{β}}_{0 , H} \\ 0 \\ {\hat{β}}_{m , H}\end{matrix} ) ) \}}^{- ( \frac{N + L - 2}{2} )} + \{ \frac{( 1 - ω ) Γ ( \frac{N + {v}_{0}}{2} )}{Γ ( \frac{{v}_{0}}{2} ) {( {v}_{0} )}^{N / 2} {π}^{N / 2}} \} {( {σ}_{0}^{2} )}^{- N / 2} {\{ det ( {I}_{N \times N} + K X {X}^{⊤} ) \}}^{- 1 / 2} \times {\{ 1 + ( \frac{1}{{v}_{0} {σ}_{0}^{2}} ) {( {y}^{( c )} )}^{⊤} {( {I}_{N \times N} + K X {X}^{⊤} )}^{- 1} {y}^{( c )} \}}^{- ( \frac{N + {v}_{0}}{2} )} .$$

Accounting for the above, weights for the two components of the posterior distribution may be Cω∫L(θ|ω, y, X)p₁(θ)dθ (for the informative component) and C(1−ω) ∫L(θ|ω, y, X)p_{F}(θ)dθ (for the flat component) respectively. The closed-form expression for the weight of the first component is:

$${ω}_{*} = \{ \frac{C ω Γ ( \frac{N + L - 2}{2} )}{Γ ( \frac{L - 2}{2} ) {( L - 2 )}^{N / 2} {π}^{N / 2}} \} {( {s}_{H}^{2} )}^{- N / 2} \{ det {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1 / 2} \times {\{ 1 + \frac{1}{( L - 2 ) {s}_{H}^{2}} {( {y}^{( c )} - X ( \begin{matrix}{\hat{β}}_{0 , H} \\ 0 \\ {\hat{β}}_{m , H}\end{matrix} ) )}^{⊤} {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1} ( {y}^{( c )} - X ( \begin{matrix}{\hat{β}}_{0 , H} \\ 0 \\ {\hat{β}}_{m , H}\end{matrix} ) ) \}}^{- ( \frac{N + L - 2}{2} )}$$

and the closed-form expression for the weight of the second component may take the form:

$$1 - {ω}_{*} = \{ \frac{C ( 1 - ω ) Γ ( \frac{N + {v}_{0}}{2} )}{Γ ( \frac{{v}_{0}}{2} ) {( {v}_{0} )}^{N / 2} {π}^{N / 2}} \} {( {σ}_{0}^{2} )}^{- N / 2} {\{ det ( {I}_{N \times N} + K X {X}^{⊤} ) \}}^{- 1 / 2} \times {\{ 1 + ( \frac{1}{{v}_{0} {σ}_{0}^{2}} ) {( {y}^{( c )} )}^{⊤} {( {I}_{N \times N} + K X {X}^{⊤} )}^{- 1} {y}^{( c )} \}}^{- ( \frac{N + {v}_{0}}{2} )} .$$

In accordance with numerous embodiments of the invention, the informative component of the mixture posterior distribution, [β₀, β_{w}, βₘ|σ², y, X] may follow a Multivariate Normal distribution with mean:

$$\begin{matrix}{β}_{*} = ( \begin{matrix}{β}_{0 , *} \\ {β}_{w , *} \\ {β}_{m , *}\end{matrix} ) \\ = ( \begin{matrix}{\hat{β}}_{0 , H} \\ 0 \\ {\hat{β}}_{m , H}\end{matrix} ) + ( \begin{matrix}{K}_{0 , H} & 0 & 0 \\ 0 & {K}_{w} & 0 \\ 0 & 0 & {K}_{m , H}\end{matrix} ) {X}^{⊤} {( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1} ( {y}^{( c )} - X ( \begin{matrix}{\hat{β}}_{0 , H} \\ 0 \\ {\hat{β}}_{m , H}\end{matrix} ) )\end{matrix}$$

and covariance matrix:

$${σ}^{2} ( \begin{matrix}{K}_{0 , H} & 0 & 0 \\ 0 & {K}_{w} & 0 \\ 0 & 0 & {K}_{m , H}\end{matrix} ) - {σ}^{2} ( \begin{matrix}{K}_{0 , H} & 0 & 0 \\ 0 & {K}_{w} & 0 \\ 0 & 0 & {K}_{m , H}\end{matrix} ) {{X}^{⊤} ( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{⊤} )}^{- 1} X ( \begin{matrix}{K}_{0 , H} & 0 & 0 \\ 0 & {K}_{w} & 0 \\ 0 & 0 & {K}_{m , H}\end{matrix} ) .$$

Additionally or alternatively, by recognizing that:

$${p}_{I} ( {σ}^{2} \vert y , X ) = \frac{{p}_{I} ( {β}_{0} , {β}_{w} , {β}_{m} , {σ}^{2} \vert y , X )}{{p}_{I} ( {β}_{0} , {β}_{w} , {β}_{m} \vert {σ}^{2} , y , X )} , \qquad ( 15 )$$

systems in accordance with several embodiments of the invention may configure the marginal posterior of the informative component to take the form:

$$[ {σ}^{2} \vert y , X ] ∼ \frac{( N + L - 2 ) {s}_{*}^{2}}{{χ}_{N + L - 2}^{2}} \qquad ( 16 )$$

$$where :$$

$${s}_{*}^{2} = \frac{1}{N + L - 2} \{ {( {y}^{( c )} − X {β}_{⋆} )}^{T} ( {y}^{( c )} − X {β}_{⋆} ) + ( L − 2 ) {s}_{H}^{2} + \frac{{( {β}_{0 , ⋆} − {\hat{β}}_{0 , H} )}^{2}}{{K}_{0 , H}} + \frac{{β}_{w , ⋆}^{2}}{{K}_{w}} + \frac{{( {β}_{m , ⋆} − {\hat{β}}_{m , H} )}^{2}}{{K}_{m , H}} \}$$

Additionally or alternatively, the flat component of the mixture posterior distribution, [β₀, β_{w}, βₘ|σ², y, X] may follow a Multivariate Normal with mean:

$${b}_{*} = ( \begin{matrix}{b}_{0 , *} \\ {b}_{w , ⋆} \\ {b}_{m , ⋆}\end{matrix} ) = {{KX}^{T} ( {I}_{N \times N} + {KXX}^{T} )}^{- 1} {y}^{( c )}$$

and covariance matrix:

$${σ}^{2} {KI}_{3 \times 3} - {σ}^{2} {K}^{2} {{X}^{T} ( {I}_{N \times N} + {KXX}^{T} )}^{- 1} X$$

Systems in accordance with some embodiments of the invention may configure the marginal posterior of σ² in the flat component to take the form:

$$[ {σ}^{2} \vert y , X ] ∼ \frac{( N + {v}_{0} ) {σ}_{0 , *}^{2}}{{χ}_{N + {v}_{0}}^{2}} \qquad ( 18 )$$

$$where :$$

$${σ}_{0 , *}^{2} = \frac{1}{N + {v}_{0}} \{ {( {y}^{( c )} - {Xb}_{*} )}^{T} ( {y}^{( c )} - {Xb}_{*} ) + {v}_{0} {σ}_{0}^{2} + \frac{{b}_{0 , *}^{2}}{K} + \frac{{b}_{w , *}^{2}}{K} + \frac{{b}_{m ,}^{2}}{K} \}$$

In accordance with a number of embodiments of the invention, formulae for the marginal posterior means and variances of β_{w} (e.g., under the additive mixture prior and/or the multiplicative mixture prior) in the case of fixed weights may be derived as a consequence of the previous expressions of the conditional and marginal posterior distributions of the parameters. These formulae may, additionally or alternatively, be applicable to the case of random weights. The marginal posterior mean of β_{w} (i.e., the treatment effect) may be calculated according to:

$$( 19 )$$

$$\begin{matrix}𝔼 \{ ( \begin{matrix}{β}_{0} \\ {β}_{w} \\ {β}_{m}\end{matrix} ) \vert ω , y , X \} = 𝔼 [ 𝔼 \{ ( \begin{matrix}{β}_{0} \\ {β}_{w} \\ {β}_{m}\end{matrix} ) \vert {σ}^{2} , Z , ω , y , X \} \vert ω , y , X ] \\ = \begin{matrix}{ω}_{*} ( \begin{matrix}{\hat{β}}_{0 , H} \\ 0 \\ {\hat{β}}_{m , H}\end{matrix} ) + {ω}_{*} ( \begin{matrix}{K}_{0 , H} & 0 & 0 \\ 0 & {K}_{w} & 0 \\ 0 & 0 & {K}_{m , H}\end{matrix} ) {X}^{T} ( {I}_{N \times N} + X ( \begin{matrix}{K}_{0 , H} & 0 & 0 \\ 0 & {K}_{w} & 0 \\ 0 & 0 & {K}_{m , H}\end{matrix} ) \\ {{X}^{T} )}^{- 1} ( {y}^{( c )} - X ( \begin{matrix}{\hat{β}}_{0 , H} \\ 0 \\ {\hat{β}}_{m , H}\end{matrix} ) ) ( - {ω}_{*} ) {{KX}^{T} ( {I}_{N \times N} + {KXX}^{T} )}^{- 1} {y}^{( c )}\end{matrix}\end{matrix}$$

while the marginal posterior variance of β_{w} can be calculated according to the law of total variance:

$$Var \{ ( \begin{matrix}{β}_{0} \\ {β}_{w} \\ {β}_{m}\end{matrix} ) \vert ω , y , X \} = 𝔼 [ Var \{ ( \begin{matrix}{β}_{0} \\ {β}_{w} \\ {β}_{m}\end{matrix} ) \vert {σ}^{2} , Z , ω , y , X \} \vert ω , y , X ] + Var [ 𝔼 \{ ( \begin{matrix}{β}_{0} \\ {β}_{w} \\ {β}_{m}\end{matrix} ) \vert {σ}^{2} , Z , ω , y , X \} \vert ω , y , X ] . \qquad ( 20 )$$

Moreover, the first term in this summation may be expanded as:

$$𝔼 [ Var \{ ( \begin{matrix}{β}_{0} \\ {β}_{w} \\ {β}_{m}\end{matrix} ) \vert {σ}^{2} , Z , ω , y , X \} \vert ω , y , X ] = {ω}_{*} ( \frac{( N + L - 2 ) {s}_{*}^{2}}{N + L - 4} ) \{ ( \begin{matrix}{K}_{0 , H} & 0 & 0 \\ 0 & {K}_{w} & 0 \\ 0 & 0 & {K}_{m , H}\end{matrix} ) - ( \begin{matrix}{K}_{0 , H} & 0 & 0 \\ 0 & {K}_{w} & 0 \\ 0 & 0 & {K}_{m , H}\end{matrix} ) {{X}^{T} ( {I}_{N \times N} + X ( \begin{matrix}{K}_{0 , H} & 0 & 0 \\ 0 & {K}_{w} & 0 \\ 0 & 0 & {K}_{m , H}\end{matrix} ) {X}^{T} )}^{- 1} X ( \begin{matrix}{K}_{0 , H} & 0 & 0 \\ 0 & {K}_{w} & 0 \\ 0 & 0 & {K}_{m , H}\end{matrix} ) \} + ( 1 - {ω}_{*} ) ( \frac{( N + {v}_{0} ) {σ}_{0 ,}^{2}}{N + {v}_{0} - 2} ) \{ K {I}_{3 \times 3} - {K}^{2} {{X}^{T} ( {I}_{N \times N} + {KXX}^{T} )}^{- 1} X \} \qquad ( 21 A )$$

while the second term in the summation can be expanded as:

$$Var [ 𝔼 \{ ( \begin{matrix}0 \\ {β}_{w} \\ {β}_{m}\end{matrix} ) \vert {σ}^{2} , Z , ω , y , X \} \vert ω , y , X ] = \begin{matrix}{ω}_{*} ( 1 - {ω}_{*} ) \times [ ( \begin{matrix}{\hat{β}}_{0 , H} \\ 0 \\ {\hat{β}}_{m , H}\end{matrix} ) + ( \begin{matrix}{K}_{0 , H} & 0 & 0 \\ 0 & {K}_{w} & 0 \\ 0 & 0 & {K}_{m , H}\end{matrix} ) {X}^{T} ( {I}_{N \times N} + \\ \begin{matrix}{X ( \begin{matrix}{K}_{0 , H} & 0 & 0 \\ 0 & {K}_{w} & 0 \\ 0 & 0 & {K}_{m , H}\end{matrix} ) {X}^{T} )}^{- 1} ( {y}^{( c )} - X ( \begin{matrix}{\hat{β}}_{0 , H} \\ 0 \\ {\hat{β}}_{m , H}\end{matrix} ) ) {KX}^{T} ( {I}_{N \times N} + \\ \begin{matrix}{{KXX}^{T} ) {y}^{( c )} ]}^{- 1} [ ( \begin{matrix}{\hat{β}}_{0 , H} \\ 0 \\ {\hat{β}}_{m , H}\end{matrix} ) + ( \begin{matrix}{K}_{0 , H} & 0 & 0 \\ 0 & {K}_{w} & 0 \\ 0 & 0 & {K}_{m , H}\end{matrix} ) {X}^{T} ( {I}_{N \times N} + X ( \begin{matrix}{K}_{0 , H} & 0 & 0 \\ 0 & {K}_{w} & 0 \\ 0 & 0 & {K}_{m , H}\end{matrix} ) \\ {{{X}^{T} )}^{- 1} ( {y}^{( c )} - X ( \begin{matrix}{\hat{β}}_{0 , H} \\ 0 \\ {\hat{β}}_{m , H}\end{matrix} ) ) - {KX}^{T} {( {I}_{N \times N} + {KXX}^{T} )}^{- 1} {y}^{( c )} ]}^{T} .\end{matrix}\end{matrix}\end{matrix}$$

Following these calculations, the marginal posterior variance may be (2,2) entry of the matrix obtained via the sum of these two terms.

Systems implemented in accordance with many embodiments of the invention may sample from the joint posterior distribution p(θ,ω|y, X) with samplers including but not limited to Gibbs samplers.

To obtain draws from p(θ, ω|y, X), systems can iterate multiple times between drawing a vector of parameters from p(θ|ω, y, X) conditional on a previous draw of ω, and drawing ω from p(ω|θ, y, X) conditional on the previously drawn θ. The Gibbs sampler, after initializing an initial weight parameter, may utilize the following steps for iteration j=1,2, . . . :

(1) Calculate ω, based on ω^{(j-1)}.

(2) Draw Z^{(j)}˜Bernouilli(ω_{*}).

(3) If Z^{(j)}=1→Draw from

$$[ {σ}^{2} \vert y , X ] ∼ \frac{( N + L - 2 ) {s}_{x}^{2}}{{χ}_{N + L - 2}^{2}} ;$$

and draw from the informative posterior component [β₀, β_{w}, βₘ|σ², y, X] conditional on the drawn σ².

(4) If Z^{(j)}=0→Draw from

$$[ {σ}^{2} \vert y , X ] ∼ \frac{( N + {v}_{0} ) {σ}_{0 , *}^{2}}{{χ}_{N + {v}_{0}}^{2}} ;$$

and draw from the flat posterior component [β₀, β_{w}, βₘ|σ², y, X] conditional on the drawn σ².

(5) Draw ω^{(j)} via the Probability Integral Transform applied to the cumulative distribution function of p(ω|θ, y, X) as obtained by numerical integration, where the previously drawn θ are conditioned upon in this distribution.

In accordance with multiple embodiments of the invention, Bayesian PROCOVA may also be used to produce a Control Arm Sample Size Reduction. This may be especially effective for the case of fixed weights. The Food and Drug Administration's Guidance for the Use of Bayesian Statistics in Medical Device Clinical Trials (incorporated by reference in its entirety) provides a simple formula for the effective sample size (ESS) of a Bayesian analysis:

$$ESS = \frac{{NV}_{1}}{{V}_{2}} ,$$

where V₁ is the posterior variance of the parameter of interest (e.g, β_{w}) that is obtained from an analysis without using an informative prior distribution (i.e., PROCOVA under the standard flat prior), and V₂ is the posterior variance of the same parameter under the informative prior distribution (i.e., Bayesian PROCOVA). Systems in accordance with multiple embodiments may have closed-form expressions for the V₁ and V₂ variances in the case of fixed weights, and so can calculate the effective sample size reduction according to

$$E S S - N = N ( \frac{{V}_{1}}{{V}_{2}} - 1 ) .$$

### 3. Bayesian PROCOVA v.2

Systems and methods implemented in accordance with several embodiments of the invention may incorporate variations on the Bayesian PROCOVA framework. The system described in this section, known as Bayesian PROCOVA v.2.0, may run on the primary assumption that large historical datasets should generally be discounted when determining priors. Specifically, in cases where historical data includes significant additional bias, the larger the dataset, the greater the possibility that new trial data is drowned in the Bayesian PROCOVA calculations. Moreover, the historical data is especially likely to influence type-I error rates. As such, this modification may have benefits including but not limited to aligning with regulatory guidance on Bayesian methodology; and accounting for anticipated changes (i.e., “offset”) in the bias of the DTG predictions (e.g., β₀) for instances where historical datasets are used for prior determination (compared to the trial(s) used for Bayesian analysis and/or posterior determination).

Systems and methods in accordance with many embodiments of the invention may modify mixture priors, including but not limited to the additive (and/or multiplicative) mixture priors through setting (e.g., user-specified) prior hyperparameters to control the type-I error rate for prospective RCTs within ranges of potential offsets. As such, implementations of Bayesian PROCOVA v.2.0 may depend on user-specified parameters including but not limited to an estimate of the variance of the digital twin offset(s) (V_{Δ}); and a discount factor applied to all components of the informative priors (γ). In accordance with many embodiments of the invention, the ideal scenarios may therefore be when the digital twin offset is at a minimum (e.g., V_{Δ}=0) and/or when the discount factor is constant.

In accordance with several embodiments of the invention, the digital twin offset may describe the difference between the bias of the digital twins on the historic and trial (control arm) data. For example, when the digital twin bias in the historical data is 1.3 points, and the bias in the control arm of the trial is 1.7 points, a corresponding offset will be 1.7-1.3=0.4 points. In many embodiments of the invention, the digital twin offset may effectively measure how much worse DTGs may perform in a prospective RCT in comparison to a historical cohort used in its evaluation.

One (recommended) method to obtain values for V_{A} is bootstrap sampling on the historical data, wherein random cohorts can be repeatedly drawn from the historical data and the variance of the digital twin offset may be computed across draws. Another method (the “transport” method) may use an analytic estimate of the variance of the digital twin offset and/or include assumptions about the consistency of parameters across historical and trial datasets (in addition to generative linear model validity).

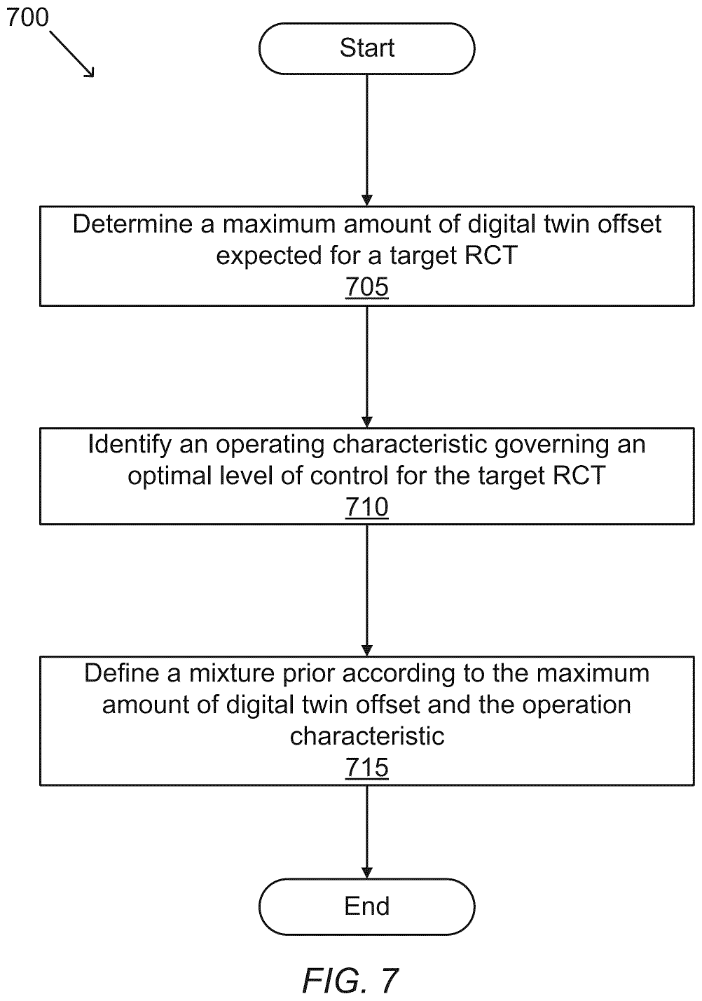

A process for setting prior hyperparameters in accordance with multiple embodiments of the invention is illustrated in FIG. 7. Process 700 determines (705) a maximum amount of digital twin offset expected for a target RCT (i.e., V_{Δmax}). In accordance with some embodiments of the invention, the maximum digital twin offset may be determined (705) based on the expected variance of the digital twin offset. For example, in many embodiments of the invention, the maximum digital twin offset may follow the formula V_{Δmax}=4*V_{Δ}^{1/2}. In accordance with certain embodiments, a maximum digital twin offset can be determined on a subjective basis to account for qualitative expectations (e.g., for novel sources of digital twin bias). Additionally or alternatively, the maximum offset parameter (V_{Δmax}) might be modified before being used in the prior. For example, an additional term (including but not limited to a constant and/or alternate variable) may be added to the maximum offset parameter.

Process 700 identifies (710) an operating characteristic governing an optimal level of control for the target RCT. In accordance with several embodiments of the invention, a maximum type-I error rate may be the operating characteristic. In such cases, a maximum type-I error rate of 10% may be an effective default. Additionally or alternatively, process 700 may identify operating characteristics including but not limited to the average type-I error rate, the average treatment effect bias, and/or the maximum treatment effect bias.

Process 700 defines (715) a mixture prior according to the maximum amount of digital twin offset and the operation characteristic. In accordance with many embodiments of the invention, defining the mixture prior may include (but is not limited to) identifying a single set of prior hyperparameter values to be used in trial planning and analysis in order to control the type-I error rate over the range of potential offsets.

While specific processes for defining system priors are described above in FIG. 7, any of a variety of processes can be utilized to adjust for type-I error and/or historical control dataset size. In certain embodiments, steps may be executed or performed in any order or sequence not limited to the order and sequence shown and described. In many embodiments, some of the above steps may be executed or performed substantially simultaneously where appropriate or in parallel to reduce latency and processing times. In several embodiments, one or more of the above steps may be omitted.

In accordance with numerous embodiments of the invention, one especially important hyperparameter value may be the discount factor (γ). The discount factor, in accordance with various embodiments of the invention, may discount the size of the historical data (used in training) relative to the planned RCT enrollment. In accordance with multiple embodiments of the invention, the minimum discount factor may be set to be the maximum between 1 and

$$\frac{{N}_{H}}{{N}_{C}}$$

(where N_{H} is the size of the historical dataset and N_{C} is the size of the control arm in the trial). Additionally or alternatively, in accordance with some embodiments of the invention, the discount factor may be determined independently of the digital twin offset value(s).

In accordance with numerous embodiments of the invention, a mixture prior distribution of the unknown model parameters can be defined based on the above values. In some embodiments, the mixture prior distribution may replace the additive mixture prior disclosed above. In several embodiments, the mixture prior distribution may replace one component (e.g., the informative component) of the additive mixture prior. Nevertheless, the mixture prior may be defined as a combination of two probability distributions:

$$β \vert {σ}^{2} ∼ 𝒩 ( {\hat{β}}_{H} , {σ}^{2} \tilde{K} ( γ ) ) , {σ}^{2} ∼ Scaled - i n v - {χ}^{2} ( \frac{{N}_{H} - 2}{γ} , {s}_{H}^{2} )$$

where \tilde{K}(γ), the proportionality constants governing the prior proportionality constants governing the prior variances of β₀, β_{w}, and βₘ are based, in part, on the discount factor and/or (maximum) digital twin offset, as:

$${\tilde{K}}_{0 , H} ( {γ}^{r} ) = \frac{1}{{N}_{c}} [ \frac{1}{2} \sqrt{{r}_{A}^{2} + 4 \frac{{V}_{Δ max}^{2}}{{σ}_{P}^{2} {x}_{c}^{2}}} + \frac{1}{2} {r}_{A} - 1 ] , = \frac{γ}{{N}_{H}} + \frac{{V}_{Δ}}{{S}_{H}^{2}}$$

$${\tilde{K}}_{ω} \to ∞$$

$${\tilde{K}}_{m , H} ( γ ) = \frac{γ}{{N}_{H}} + \frac{1}{{s}_{m , H}^{2}} ,$$

where N_{C} represents the expected size of the trial's control arm, N_{H} represents the size of the historical dataset, r_{A} represents the expected fraction randomized to treatment for the trial, σ_{P}² represents the expected variance (as determined using PROCOVA), and x_{c} represents a constant defined from the target maximum type-I error rate. In accordance with multiple embodiments of the invention xc may represent the corresponding effect size. Additionally or alternatively, for a 10% type-I error rate with a nominal decision rule significance of 0.05, the effect size may be set to x_{c}=0.6523).

Systems and techniques for applying Bayes' rule to prognostic models used in the assessment of experiment uncertainty, are not limited to use for randomized controlled trials. Accordingly, it should be appreciated that applications described herein can be implemented outside the context of generative model architecture and in contexts unrelated to RCTs. Moreover, any of the systems and methods described herein with reference to FIGS. 3-7 can be utilized within any of the processes described above.

### III. Systems for Determining Treatment Effects

### A. Treatment Analysis System

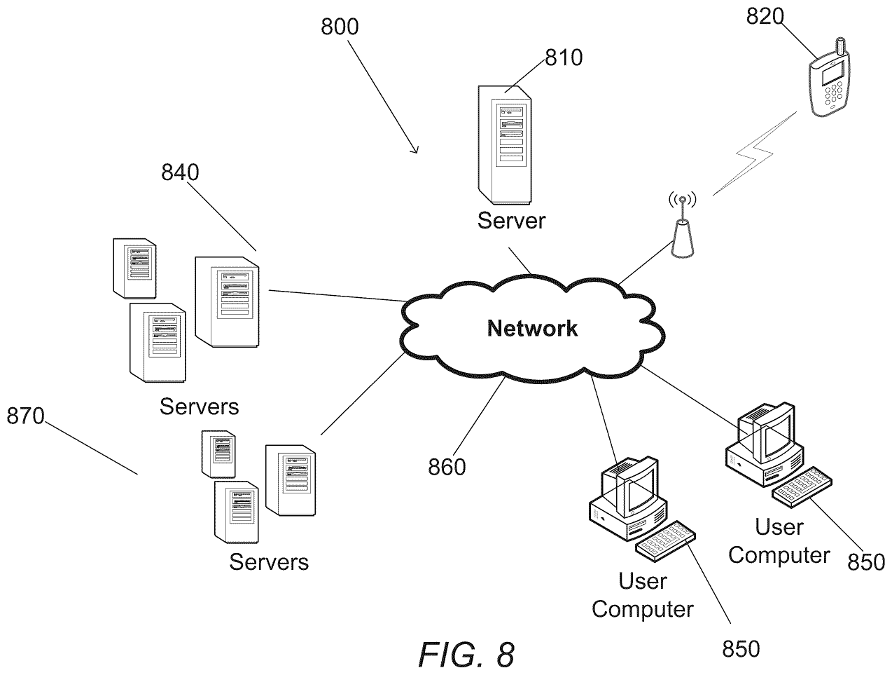

An example of a treatment analysis system that determines treatment effects in accordance with some embodiments of the invention is illustrated in FIG. 8. Network 800 includes a communications network 860. The communications network 860 is a network such as the Internet that allows devices connected to the network 860 to communicate with other connected devices. Server systems 810, 840, and 870 are connected to the network 860. Each of the server systems 810, 840, and 870 is a group of one or more servers communicatively connected to one another via internal networks that execute processes that provide cloud services to users over the network 860. One skilled in the art will recognize that a treatment analysis system may exclude certain components and/or include other components that are omitted for brevity without departing from this invention.

For purposes of this discussion, cloud services are one or more applications that are executed by one or more server systems to provide data and/or executable applications to devices over a network. The server systems 810, 840, and 870 are shown each having three servers in the internal network. However, the server systems 810, 840 and 870 may include any number of servers and any additional number of server systems may be connected to the network 860 to provide cloud services. In accordance with various embodiments of this invention, treatment analysis systems in accordance with various embodiments of the invention may be provided by a process being executed on a single server system and/or a group of server systems communicating over network 860.

Users may use personal devices 880 and 820 that connect to the network 860 to perform processes that determine treatment effects in accordance with various embodiments of the invention. In the shown embodiment, the personal devices 880 are shown as desktop computers that are connected via a conventional “wired” connection to the network 860. However, the personal device 880 may be a desktop computer, a laptop computer, a smart television, an entertainment gaming console, and/or any other device that connects to the network 860 via a “wired” connection. The mobile device 820 connects to network 860 using a wireless connection. A wireless connection is a connection that uses Radio Frequency (RF) signals, Infrared signals, and/or any other form of wireless signaling to connect to the network 860. In FIG. 8, the mobile device 820 is a mobile telephone. However, mobile devices 820 may be mobile phones, Personal Digital Assistants (PDAs), tablets, smartphones, and/or any other type of device that connects to network 860 via wireless connection without departing from this invention.

As can readily be appreciated the specific computing system used to determine treatment effects is largely dependent upon the requirements of a given application and should not be considered as limited to any specific computing system(s) implementation.

### B. Treatment Analysis Element

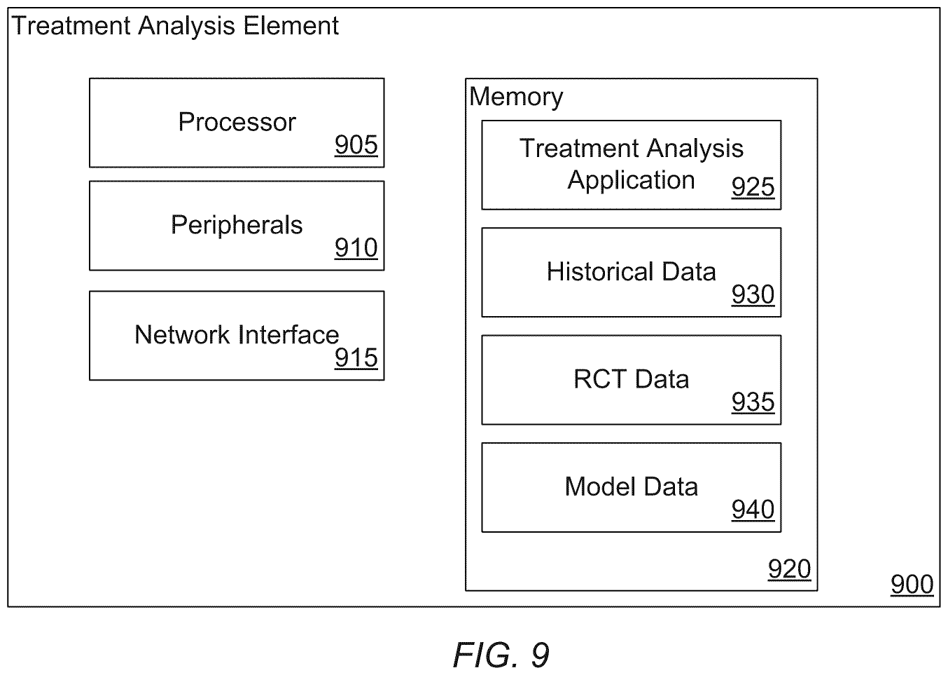

An example of a treatment analysis element that executes instructions to perform processes that determine treatment effects in accordance with various embodiments of the invention is illustrated in FIG. 9. Treatment analysis elements in accordance with many embodiments of the invention can include (but are not limited to) one or more of mobile devices, cloud services, and/or computers. Treatment analysis element 900 includes processor 905, peripherals 910, network interface 915, and memory 920. One skilled in the art will recognize that a treatment analysis element may exclude certain components and/or include other components that are omitted for brevity without departing from this invention.

The processor(s) 905 can include (but is not limited to) a processor, microprocessor, controller, and/or a combination of processors, microprocessor, and/or controllers that perform instructions stored in the memory 920 to manipulate data stored in the memory. Processor instructions can configure processors 905 to perform processes in accordance with certain embodiments of the invention.

Peripherals 910 can include any of a variety of components for capturing data, such as (but not limited to) cameras, displays, and/or sensors. In a variety of embodiments, peripherals can be used to gather inputs and/or provide outputs. Treatment analysis element 900 can utilize network interface 915 to transmit and receive data over a network based upon the instructions performed by processor 905. Peripherals 910 and/or network interfaces 915 in accordance with many embodiments of the invention can be used to gather data that can be used to determine treatment effects.

Memory 920 includes a treatment analysis application 925, historical data 930, RCT data 935, and model data 940. Treatment analysis applications in accordance with several embodiments of the invention can be used to determine treatment effects of an RCT, to design an RCT, and/or determine decision rules for treatments. Processors 905 implemented in accordance with numerous embodiments of the invention may be configured to process input data according to instructions stored in memory 920. Memory 920 may include but are not limited to hard disk drives, nonvolatile memory, and/or other non-transient storage devices. In many embodiments, memory 920 can be loaded with software code that is executable by processors to achieve certain functions. Memory 920 may exist in the form of tangible, non-transitory, computer-readable mediums configured to store instructions that cause the processor(s) 905 to perform various processes described above.

Historical data 930 in accordance with many embodiments of the invention can be used to pre-train generative models to generate potential outcomes for digital subjects and/or digital twins. In numerous embodiments, historical data 930 can include (but is not limited to) control arms from historical control arms, patient registries, electronic health records, and/or real-world data. In many embodiments, predictions from generative models can be compared to historical data 930 that was not used to train the models in order to obtain prior distributions capturing how well the predictions generalize to new populations.

In some embodiments, RCT data 935 can include panel data collected from subjects of RCTs. RCT data 935 in accordance with a variety of embodiments of the invention can be divided into control and treatment arms based on whether subjects received a treatment. In many embodiments, RCT data 935 can be supplemented with generated subject data. Generated subject data in accordance with a number of embodiments of the invention can include (but is not limited to) digital subject data and/or digital twin data.

In several embodiments, model data 940 can store various parameters and/or weights for generative models. Model data 940 in accordance with many embodiments of the invention can include data for models trained on historical data 930 and/or trained on RCT data 935. In several embodiments, pre-trained models can be updated based on RCT data 935 to generate digital subjects.

Although a specific example of a treatment analysis element 900 is illustrated in this figure, any of a variety of treatment analysis elements can be utilized to perform processes for determining treatment effects similar to those described herein as appropriate to the requirements of specific applications in accordance with embodiments of the invention.

### C. Treatment Analysis Application

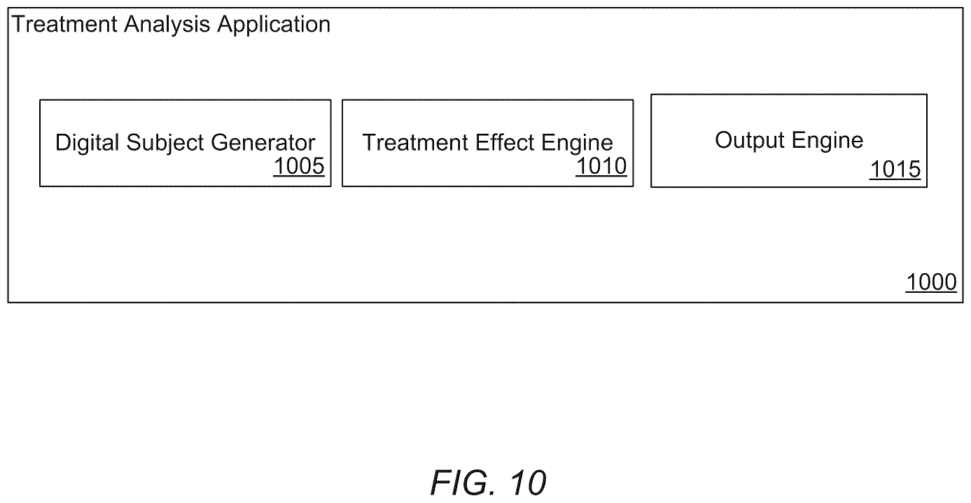

An example of a treatment analysis application for determining treatment effects in accordance with some embodiments of the invention is illustrated in FIG. 10. Treatment analysis applications 1000 includes digital subject generator 1005, treatment effect engine 1010, and output engine 1015. One skilled in the art will recognize that a treatment analysis application may exclude certain components and/or include other components that are omitted for brevity without departing from this invention.

Digital subject generators 1005 in accordance with various embodiments of the invention can include generative models that can generate digital subject and/or digital twin data. Generative models in accordance with certain embodiments of the invention can be trained to generate potential outcome data based on characteristics of an individual and/or a population. Digital subject data in accordance with several embodiments of the invention can include (but is not limited to) panel data, outcome data, etc. In several embodiments, generative models can include (but are not limited to) traditional statistical models, generative adversarial networks, recurrent neural networks, Gaussian processes, autoencoders, autoregressive models, variational autoencoders, and/or other types of probabilistic generative models.

In various embodiments, treatment effect engines 1010 can be used to determine treatment effects based on generated digital subject data and/or data from a RCT. In some embodiments, treatment effect engines 1010 can use digital subject data from digital subject generators 1005 to determine treatment effects in a variety of different applications, such as, but not limited to, comparing separate generative models based on data from the control and treatment arms of RCTs, supplementing control arms in RCTs, comparing predicted potential control outcomes with actual treatment outcomes, etc. Treatment effect engines 1010 in accordance with many embodiments of the invention can be used to determine individualized responses to treatment. In certain embodiments, treatment effect engines 1010 can determine biases of generative models of the digital subject generator and incorporate the biases (or corrections for the biases) in the treatment effect analyses.

Output engines 1015 in accordance with several embodiments of the invention can provide a variety of outputs to a user, including (but not limited to) decision rules, treatment effects, generative model biases, recommended RCT designs, etc. In numerous embodiments, output engines 1015 can provide feedback when the results of generative models of a digital subject generator 1005 diverge from the RCT population. For example, output engines in accordance with certain embodiments of the invention can provide a notification when a difference between generated control outcomes for digital twins of subjects from a control arm and their actual control outcomes exceeds a threshold.

Although a specific example of a treatment analysis application is illustrated in this figure, any of a variety of treatment analysis applications can be utilized to perform processes for determining treatment effects similar to those described herein as appropriate to the requirements of specific applications in accordance with various embodiments of the invention.

Although specific systems and methods for covariate adjustment are discussed above, many different methods of model implementation can be utilized in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for updating predictive models, the method comprising:
receiving, from at least one randomized controlled trial (RCT), RCT data, wherein the RCT data comprises information on trial subjects from control arms of the at least one RCT;
training a set of one or more generative models based on the RCT data;
defining a mixture prior distribution corresponding to unknown parameters of the set of one or more generative models, wherein the mixture prior distribution comprises:
an informative component, wherein the informative component follows an informative prior distribution defined, at least in part, on the RCT data; and
a flat component, wherein the flat component follows a flat prior distribution defined independently of the RCT data;
generating, using the set of one or more generative models, predicted panel data for a plurality of digital subjects, wherein the predicted panel data for a given digital subject comprises a plurality of predicted outcomes on at least one characteristic of the given digital subject in response to applying a treatment in a target RCT;
deriving a mixture posterior distribution corresponding to the unknown parameters of the set of one or more generative models, based on the predicted panel data; and
determining, based on at least one of the predicted panel data or the mixture posterior distribution, a set of one or more decision rules, wherein the set of one or more decision rules govern type-I estimates for the set of one or more generative models.

2. The method of claim 1, wherein:
defining the mixture prior distribution comprises computing a weighted combination of the informative component and the flat component;
the informative component has a first weight and the flat component has a second weight; and
deriving the mixture posterior distribution comprises computing an updated first weight and an updated second weight.

3. The method of claim 2, wherein:
the weighted combination is a weighted sum;
the first weight is selected from a weight parameter prior distribution that follows a Beta distribution; and
the first weight and second weight sum to 1.

4. The method of claim 1, wherein generating the predicted panel data, for each digital subject of the plurality of digital subjects, comprises:
determining, for the digital subject, at least one of:
a baseline vector, wherein the baseline vector comprises time-dependent measurements, corresponding to a specific time before a given RCT, that are based on the RCT data; or
a covariate vector, wherein the covariate vector comprises time-independent characteristics of the digital subject that are based on the RCT data; and
inputting the at least one of the baseline vector or the covariate vector into the set of one or more generative models.

5. The method of claim 4, wherein generating the predicted panel data, for each digital subject of the plurality of digital subjects, further comprises:
obtaining, from the set of one or more generative models, a control outcome distribution, wherein the control outcome distribution describes potential values for an expected outcome if the digital subject were placed in a control group for the given RCT;
determining the control outcome distribution, a score for the digital subject, wherein the score corresponds to the expected outcome; and
inputting, into the set of one or more generative models:
a randomized treatment indicator, wherein the randomized treatment indicator corresponds to whether the digital subject is part of a treatment arm in the target RCT; and
the score for the digital subject.

6. The method of claim 1, wherein:
defining the informative prior distribution is based on:
a first informative parameter selected from an Inverse Chi-Squared probability distribution, wherein the Inverse Chi-Squared probability distribution is defined based on the RCT data; and
a second informative parameter selected from a Multivariate Normal probability distribution, wherein the Multivariate Normal probability distribution is defined based on the RCT data and the first informative parameter; and
defining the flat prior distribution is based on:
a first flat parameter selected from an Inverse Chi-Squared probability distribution, wherein the Inverse Chi-Squared probability distribution is defined based on a set of input flat values; and
a second flat parameter selected from a Multivariate Normal probability distribution, wherein the Multivariate Normal probability distribution is defined based on the set of input flat values and the first flat parameter.

7. The method of claim 6, wherein the second informative parameter is further defined based on at least one of:
a discount factor, wherein the discount factor represents a discount to an amount of the RCT data used when training the set of one or more generative models; or
a maximum estimate for an offset value, wherein the offset value represents a difference between a first bias measured for the RCT data and a second bias measured for the predicted panel data.

8. The method of claim 7, wherein:
the discount factor is determined based on a population size for the plurality of digital subjects; and
the maximum estimate is linearly proportional to a square root of a variance in the offset value, wherein the variance in the offset value is determined based on bootstrap sampling of the RCT data to determine various values for the first bias.

9. The method of claim 1, wherein determining the set of one or more decision rules comprises:
estimating values for the unknown parameters, wherein:
Gibbs sampling is used for estimating the values; and
at least one of the unknown parameters relates to an effect of the treatment;
deriving an uncertainty estimate corresponding to the estimated effect of the treatment; and
determining the set of one or more decision rules based on the uncertainty estimate.

10. The method of claim 1, wherein the set of one or more generative models comprises at least one of a Conditional Restricted Boltzmann Machine, a statistical model, a generative adversarial network, a recurrent neural network, a Gaussian process, an autoencoder, an autoregressive model, or a variational autoencoder.

11. A non-transitory computer-readable medium comprising instructions that, when executed, are configured to cause a processor to perform a process for updating predictive models, the process comprising:
receiving, from at least one randomized controlled trial (RCT), RCT data, wherein the RCT data comprises information on trial subjects from control arms of the at least one RCT;
training a set of one or more generative models based on the RCT data;
defining a mixture prior distribution corresponding to unknown parameters of the set of one or more generative models, wherein the mixture prior distribution comprises:
an informative component, wherein the informative component follows an informative prior distribution defined, at least in part, on the RCT data; and
a flat component, wherein the flat component follows a flat prior distribution defined independently of the RCT data;
generating, using the set of one or more generative models, predicted panel data for a plurality of digital subjects, wherein the predicted panel data for a given digital subject comprises a plurality of predicted outcomes on at least one characteristic of the given digital subject in response to applying a treatment in a target RCT;
deriving a mixture posterior distribution corresponding to the unknown parameters of the set of one or more generative models, based on the predicted panel data; and
determining, based on at least one of the predicted panel data or the mixture posterior distribution, a set of one or more decision rules, wherein the set of one or more decision rules govern type-I estimates for the set of one or more generative models.

12. The non-transitory computer-readable medium of claim 11, wherein:
defining the mixture prior distribution comprises computing a weighted combination of the informative component and the flat component;
the informative component has a first weight and the flat component has a second weight; and
deriving the mixture posterior distribution comprises computing an updated first weight and an updated second weight.

13. The non-transitory computer-readable medium of claim 12, wherein:
the weighted combination is a weighted sum;
the first weight is selected from a weight parameter prior distribution that follows a Beta distribution; and
the first weight and second weight sum to 1.

14. The non-transitory computer-readable medium of claim 11, wherein generating the predicted panel data, for each digital subject of the plurality of digital subjects, comprises:
determining, for the digital subject, at least one of:
a baseline vector, wherein the baseline vector comprises time-dependent measurements, corresponding to a specific time before a given RCT, that are based on the RCT data; or
a covariate vector, wherein the covariate vector comprises time-independent characteristics of the digital subject that are based on the RCT data; and
inputting the at least one of the baseline vector or the covariate vector into the set of one or more generative models.

15. The non-transitory computer-readable medium of claim 14, wherein generating the predicted panel data, for each digital subject of the plurality of digital subjects, further comprises:
obtaining, from the set of one or more generative models, a control outcome distribution, wherein the control outcome distribution describes potential values for an expected outcome if the digital subject were placed in a control group for the given RCT;
determining the control outcome distribution, a score for the digital subject, wherein the score corresponds to the expected outcome; and
inputting, into the set of one or more generative models:
a randomized treatment indicator, wherein the randomized treatment indicator corresponds to whether the digital subject is part of a treatment arm in the target RCT; and
the score for the digital subject.

16. The non-transitory computer-readable medium of claim 11, wherein:
defining the informative prior distribution is based on:
a first informative parameter selected from an Inverse Chi-Squared probability distribution, wherein the Inverse Chi-Squared probability distribution is defined based on the RCT data; and
a second informative parameter selected from a Multivariate Normal probability distribution, wherein the Multivariate Normal probability distribution is defined based on the RCT data and the first informative parameter; and
defining the flat prior distribution is based on:
a first flat parameter selected from an Inverse Chi-Squared probability distribution, wherein the Inverse Chi-Squared probability distribution is defined based on a set of input flat values; and
a second flat parameter selected from a Multivariate Normal probability distribution, wherein the Multivariate Normal probability distribution is defined based on the set of input flat values and the first flat parameter

17. The non-transitory computer-readable medium of claim 16, wherein the second informative parameter is further defined based on at least one of:
a discount factor, wherein the discount factor represents a discount to an amount of the RCT data used when training the set of one or more generative models; or
a maximum estimate for an offset value, wherein the offset value represents a difference between a first bias measured for the RCT data and a second bias measured for the predicted panel data.

18. The non-transitory computer-readable medium of claim 17, wherein:
the discount factor is determined based on a population size for the plurality of digital subjects; and
the maximum estimate is linearly proportional to a square root of a variance in the offset value, wherein the variance in the offset value is determined based on bootstrap sampling of the RCT data to determine various values for the first bias.

19. The non-transitory computer-readable medium of claim 11, wherein determining the set of one or more decision rules comprises:
estimating values for the unknown parameters, wherein:
Gibbs sampling is used for estimating the values; and
at least one of the unknown parameters relates to an effect of the treatment;
deriving an uncertainty estimate corresponding to the estimated effect of the treatment; and
determining the set of one or more decision rules based on the uncertainty estimate.

20. The non-transitory computer-readable medium of claim 11, wherein the set of one or more generative models comprises at least one of a Conditional Restricted Boltzmann Machine, a statistical model, a generative adversarial network, a recurrent neural network, a Gaussian process, an autoencoder, an autoregressive model, or a variational autoencoder.
